(19) United States
(12) Patent Application Publication
(10) Pub. No.: US 2026/0074076 A1
(43) Pub. Date: Mar. 12, 2026

(54) GENERATING INTERVENTION SUCCESS PROBABILITIES AND INTELLIGENT RANKING OF SUBJECTS

(71) Applicant: Oracle International Corporation, Redwood Shores, CA (US)

(72) Inventors: Nathan Becker, Los Angeles, CA (US); Renee George, San Diego, CA (US); Christine Swisher, San Diego, CA (US); Graham Bury, Redmond, WA (US); Xerxes Beharry, Kirkland, WA (US); Benjamin Ellis, Mt. Horeb, WI (US); Jason Weinreb, New York, NY (US); Josue Martinez-Montero, Redmond, WA (US)

(73) Assignee: Oracle International Corporation, Redwood Shores, CA (US)

(21) Appl. No.: 19/316,994

(22) Filed: Sep. 2, 2025

Related U.S. Application Data
(60) Provisional application No. 63/692,017, filed on Sep. 6, 2024.
(60) Provisional application No. 63/831,451, filed on Jun. 27, 2025.

(51) Int. Cl. G16H 50/30; G16H 40/20

(52) U.S. Cl. CPC G16H 50/30; G16H 40/20

(57) ABSTRACT

The present disclosure relates to techniques for generating a ranked list of a set of subjects by predicting their potential health benefit from an intervention to prioritize subjects that may be at a risk of a negative outcome and likely to benefit from a proposed intervention. Additionally, the ranking may further account for potential cost-savings associated with early intervention to avoid acute-care utilization by applying a cost-modeling technique. The disclosed techniques may include analyzing subject-specific data, including demographic, clinical, and historical information, to compute a total net-benefit score by combining a predicted benefit probability with cost and revenue metrics. The benefit probability may be calculated using causal inference models to estimate a potential improvement in health outcomes from the proposed intervention or treatment. The disclosed techniques may further facilitate personalized subject care by dynamically updating rankings based on real-time data, enhancing clinical decision-making, and optimizing resource allocation.

### CROSS-REFERENCE TO RELATED APPLICATIONS

This application claims the benefit of and the priority to U.S. Provisional Application No. 63/692,017, filed on Sep. 6, 2024, entitled “Generating Intervention Success Probabilities and Intelligent Ranking of Subjects” and U.S. Provisional Application No. 63/831,451, filed on Jun. 27, 2025, entitled “Generating Intervention Success Probabilities and Intelligent Ranking of Subjects”. Each of these applications is hereby incorporated by reference in its entirety for all purposes.

### BACKGROUND

In modern healthcare systems, medical organizations frequently face challenges to efficiently and effectively schedule human, material and equipment resources, particularly when these resources have limited stock in the store and the decision is to be taken in nearly real-time to plan and administer treatments for critically ill patients. Approximately 15% of patients who are discharged from hospitals end up being readmitted in the emergency of the hospital within 30 days. These emergency readmissions, which are generally unplanned, may have a significant impact on hospital resources, and hence is a serious concern for the management of the hospital. Clinicians or care managers may prioritize these patients and may plan routine follow-up visits to reduce unplanned readmissions. Additionally, clinicians may be asked to review the health condition of patients at the time of discharge and then assess their risks to prioritize them for receiving priority care, if readmitted. The priority care may involve scheduling a follow-up visit, ordering additional lab investigations, or planning treatments to be given if readmitted in the emergency of the hospital. This complex decision-making process is generally left to the subjective judgement of an individual clinician, who cannot reliably determine the likelihood of various adverse outcomes based on the limited amount of patients' data.

Moreover, resource scheduling and allocation may become significantly challenging when hospitals have a limited supply of medications, a small amount of medical equipment, and a small number of nursing staff in the emergency department. It is possible that care providers may assess the severity (or risk) of individual patients' health conditions that are directly under their treatment in a reactive manner. The reactive methods often do not consider data driven evidence in the decision making and hence lead to an inefficient, and sometimes ineffective, healthcare delivery system, resulting in missing a significant percentage of high-risk patients that need prioritized timely healthcare services.

### SUMMARY

Some embodiments of the present disclosure relate to techniques for generating a ranked list of a set of subjects by predicting their potential health benefit from an intervention to prioritize the subjects that may be at a risk of a negative outcome and likely to benefit from a proposed intervention. The ranking of the set of subjects may include identifying a set of subjects flagged for engagement in a potential communication workflow by accessing subjects' data from electronic health record (EHR). The subject's data may include a plurality of confounding features, which are variables that may influence both treatment assignment and its outcome, potentially affecting the estimation of causal effects. These confounding features may include demographic attributes (e.g., age, gender, socioeconomic status), comorbid conditions, prior treatments, genetic predispositions, and lifestyle factors such as smoking or exercise habits.

The disclosed techniques involve leveraging subject-specific data, including demographic, clinical, and historical health records, to generate intervention benefit probabilities using causal inference techniques. The system may identify causal relationships between subject attributes and past treatment outcomes by analyzing large-scale observational data. These relationships may allow for estimating three distinct benefit probabilities: the likelihood of achieving a positive outcome if the intervention is performed, the likelihood of a positive outcome without the intervention, and the probability that the subject would receive the intervention based on their current health state. The estimated benefit probabilities may be combined into a net-benefit bounds that reflect the overall effectiveness of medical interventions, serving as a basis for data-driven clinical decision-making. The net-benefit bounds may include an upper and lower bound that may represent a range of potential benefit estimates.

According to the disclosed techniques, the system may leverage one or more causal models for estimating the benefit probabilities. These models may be trained on historical intervention assignments and corresponding health outcomes, incorporating confounding adjustments to improve estimation accuracy. The training process of the causal models may involve learning subject-treatment relationships (also referred herein as causal relationships) from structured electronic health records (EHRs) and real-world clinical studies. By modeling intervention effects using counterfactual reasoning, the system may allow for more precise estimation of treatment impact while accounting for patient heterogeneity. According to some aspects, the causal models may include directed acyclic graphs (DAGs), inverse probability weighting (IPW), uplift modeling, propensity score matching (PSM), structural causal modeling (SCM), and/or structural equation modeling (SEM).

According to the disclosed techniques, the net benefit bounds (or PNS bounds) may further be used to estimate net-cost saving bounds by combining the predicted net-benefit bounds with cost savings associated with the intervention. The cost savings may include an amount associated with performing the intervention, an amount associated with an acute-care utilization, and a yield or revenue associated with closing care gaps as a result of performing the intervention. Resultantly, the estimated net-cost saving bounds may represent a collective benefit of a healthcare provider and the subject. By incorporating estimates of the net-benefit bounds, the cost savings may aid in prioritizing interventions for subjects where the expected impact may be most meaningful, assisting the care givers or clinicians to direct clinical, operational, or strategic resources toward cases with the highest potential for improvement. By doing so, the system may prioritize subjects that may be most likely to benefit from the intervention while also allocating resources (e.g., hospital staff, funding, and medical bandwidth) in a way that may maximize both clinical impact and financial sustainability.

The system may gather historical claims data from the EHR including historical costs of a preventative action (e.g., a cost of a follow-up appointment), historical cost of an emergency department visit, and historical revenue generated from closing care gaps (performing necessary screenings, vaccinations, or treatments during the appointments). Based on the historical claims data, the system may leverage various ML models to predict the cost for the preventative treatment or intervention. The ML models may be configured to predict an average cost for the preventative intervention, the cost of an ED visit or an ACU, and an expected revenue generated from closing care gaps for the patient as a result of the intervention being carried out. Based on the predicted cost savings and the net-benefit bounds (i.e., the upper and lower PNS bounds), a net-cost saving bound may be calculated, using a mathematical function. The mathematical function may involve summing up the predicted costs and multiplying with the net-benefit bounds, resulting in bounds on the net-cost savings probability. The predicted net-cost saving bounds may quantify or represent the overall financial and health impact of administering the intervention, reflecting the potential savings in healthcare expenditures while also accounting for the health benefits of the subject through preventative care or the intervention.

The disclosed techniques may further involve ranking a set of subjects based on the computed net-cost saving bounds to prioritize individuals for healthcare interventions. In some aspects, to generate the ranked list, each subject may be assigned a first rank based on the upper bound of the net-cost saving probability using a standard competition ranking method that assigns the subjects with the same upper bound a same rank, and the next rank may be incremented accordingly. Similarly, a second rank may be assigned based on the lower bound of the benefit probability. Since these two ranks may not always align, they may be averaged to generate a combined rank score that reflects both upper and lower bounds.

Finally, the system generates the ranked list by sorting subjects based on their assigned relative rank scores, allowing for an optimized prioritization strategy that may enhance healthcare resource allocation and intervention planning. The output and/or the ranked list may be presented on a device (e.g., a tablet, a laptop etc.) of the user or a care manager.

For the subjects with a high rank in the ranked list, the system enables the care managers to focus resources and attention on subjects who would benefit the most from the intervention (i.e., scheduling an appointment). For example, if the user is a care manager of a healthcare provider, the system may assist in determining which subjects should be prioritized for follow-up appointments based on their health status. The goal is to rank subjects based on two key factors: the potential benefit they may receive from a predefined intervention—which also encompasses the likelihood of preventing a negative outcome—and the potential cost-savings associated with taking early action to improve the subject's outcome. This ranking may help the care managers to prioritize subjects who are both at high risk and most likely to benefit, ultimately supporting more effective and targeted healthcare decision-making. Continually analyzing and updating the rankings of the subjects based on their real-time data enables healthcare providers to make informed decisions regarding subjects' care and to preemptively provide healthcare services to the subjects, with potential health crises, on a priority.

The system may continuously monitor and process real-time data, to enable accurate and up-to-date subjects' rankings, which may be recalculated on a daily basis. For example, if a subject was previously ranked high based on earlier health data, but newly updated EHR data indicates improved health, their ranking may be adjusted accordingly. Conversely, if new information suggests a decline in health, their ranking may increase to reflect the greater need for intervention. Since rankings may be recomputed daily using the latest available EHR data, they may dynamically update to reflect the most current subject status.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an exemplary system for executing a method to generate a ranked list based on subjects' records in accordance with some aspects of the present disclosure.

FIG. 2 shows an exemplary overview of a subject ranking system in accordance with some aspects of the present disclosure.

FIG. 3 shows an exemplary block diagram of a benefit predictor for estimating a net benefit bound in accordance with some aspects of the present disclosure.

FIG. 4 shows an exemplary block diagram of a cost prediction model to predict net-cost savings in accordance with some aspects of the present disclosure.

FIG. 5 shows a training process of a pair-wise surrogate model for analyzing feature importance of the subjects during ranking in accordance with some aspects of the present disclosure.

FIG. 6 shows a training process of causal models for predicting the net-benefit bounds by using a double-stratified sampling technique.

FIG. 7 illustrates an exemplary overview of the subject ranking system at inference, incorporating a bias adjustment unit to calibrate output of the causal model in accordance with some aspects of the present disclosure.

FIG. 8 shows a selection diagram detailing the transportability process from a double-stratified training domain to a full-data (real-world) domain for probability estimates.

FIG. 9 shows an example flowchart of a system for generating intervention success probabilities and ranking the subjects in accordance with some aspects of the present disclosure.

FIG. 10 illustrates a simplified diagram of an example distributed system for a cloud hosting the subject ranking system.

FIG. 11 is a simplified block diagram of a cloud-based system environment in which various services of a server of FIG. 10 may be offered as cloud services, in accordance with some aspects of the present disclosure.

FIG. 12 illustrates an exemplary computer system to implement some aspects of the present disclosure.

### DETAILED DESCRIPTION

Some embodiments of the present disclosure relate to generation of a ranked list of subjects in an order of effectiveness of a treatment or an intervention received from a user or a care manager. A rank of a subject in the sorted list may be based on parameters that may include a net-benefit score quantifying a potential improvement in health status of the subject as a result of the intervention, and a likelihood of a positive outcome without the (timely) intervention, and the probability representing the likelihood that the subject would receive the intervention under standard care procedures, based on their characteristics or health status. The net-benefit score may be predicted using a subject ranking system based on a set of records of the subject (e.g., a patient) that may be related to the intervention. The subject ranking system may perform an evaluation of the impact of the intervention for each subject, and rank the individual subjects based on the benefit from the intervention.

The set of records of the subject may include data such as personal information and medical history (e.g., lab reports, inpatient or outpatient clinical notes etc.), and visit or encounter type (e.g., specific disease or problem, specific department such as cardiology, urology etc.). In some instances, the data may further include symptoms, problems, or clinical conditions that may automatically be extracted from a message, communication, or an email of the subject that may be sent for a virtual consultation.

According to some aspects, the user (e.g., care managers, healthcare professionals, clinicians, physicians, or nurses) may record health information of the subject during various encounters. Each record can be stored in a database with an identifier and a metadata. The metadata may include information about the subject, author of the record, encounter type, record type etc. The database can be an electronic health record (EHR) database that may include an electronic medical record (EMR) database, a cloud-based database, a local database of an organization and the like.

In most clinical scenarios, it is impossible to have subject data reflecting both scenarios (receiving and not receiving a same intervention) for the same subject at the same time. This limitation may introduce uncertainty in fully understanding the consequences of different clinical actions. For this purpose, causal inference models may be employed to infer what the outcome would have been if the opposite action had been taken. The causal inference model is a framework used to describe and understand the cause-and-effect relationships between variables within a system. These models aim to explain how certain variables influence others, providing a structured way to predict outcomes and evaluate the impact of different interventions or changes. This allows users to form a more comprehensive understanding of the relative benefit of either performing or withholding an action. By applying these techniques, the subject ranking system can construct a comparative picture of the benefits and potential risks associated with various treatments, facilitating informed decision-making.

For example, consider an intervention request labeled “Schedule a Follow-up Appointment” for an individual at risk of experiencing an acute-care utilization (ACU), such as an emergency department (ED) visit or an ED visit followed by hospitalization (ED+admission). The goal of the patient ranking is to calculate a benefit score that reflects how much a subject encounter might reduce the likelihood of an ACU. This assessment is critical in guiding clinical prioritization and resource allocation, particularly when resources are limited. By leveraging causal inference models, the system predicts the likely outcomes under both scenarios—whether the subject encounter occurs or does not occur—helping to quantify the potential benefit of the intervention in reducing the patient's risk of an ACU.

To achieve this, the system assembles an intervention dataset (also referred herein as intervention data), which includes three core variables essential to the causal inference model. These variables are the action indicator (whether the patient encounter occurred or not), the outcome indicator (whether the patient avoided an ACU), and confounding features (patient-specific attributes such as age, comorbidities, and treatment history, which may affect both the decision to intervene and the resulting outcome). These confounding features may be extracted from the electronic health record (EHR), enabling the system to consider all relevant subject factors influencing the intervention, and its outcome in the analysis. According to some aspects, intervention data may also include data recorded for each past interaction with subjects as to whether an intervention was identified as relevant, whether an intervention was attempted to be applied to the subject, whether the intervention was received by the subject, and whether a successful outcome was achieved for the intervention. Labeled data may also be generated based on past data by a model for generating intervention data.

According to some embodiments, three distinct supervised learning binary classifiers (also referred herein as causal inference models or causal models) may be trained on the intervention data and the subject data from the EHR, each responsible for modeling one of the key variables, allowing the full predictive model to be assembled. Instead of computing an exact benefit score or value, the models may estimate upper and lower bounds of the net-benefit probability (also referred herein as the net-benefit bounds), providing a range for a probability of necessary and sufficient (PNS). By computing bounds rather than a single point estimate, the system becomes more resilient to noise and missing data, allowing for greater flexibility in real-world clinical scenarios. This bounded approach may enhance the robustness of the subject ranking system, assisting the user to prioritize subjects based on reliable estimates of potential benefit.

According to some aspects of the present disclosure, the system may incorporate business logic related to cost savings into the ranking system to enhance the prioritization of healthcare interventions while also integrating financial consideration into the ranking process. The inclusion of cost saving elements in the final ranking assists care managers in directing clinical, operational, and strategic resources toward cases with the highest potential for improvement. By doing so, the ranking system may identify and prioritize subjects who are most likely to benefit from the intervention while ensuring the efficient allocation of resources, such as hospital staff, funding, and medical bandwidth, in a way that maximizes both clinical impact and operational sustainability.

To implement this, the system may leverage a cost prediction model configured to estimate three key financial metrics or costs: an average cost of the preventative intervention (e.g., the follow-up appointments), an expected cost of acute-care utilization (ACU) (such as an emergency department (ED) visit or a hospitalization due to lack of timely intervention), and an expected revenue incurred from closing care gaps by performing the (timely) intervention (e.g., performing necessary screenings, vaccinations, or treatments during scheduled visits). The system may gather historical claims data from electronic health records (EHRs), including past expenditures related to preventative actions and acute-care utilization, along with revenue data from successfully closing care gaps. Based on these historical patterns, the cost prediction model may estimate the aforementioned financial metrics. These predicted cost and revenue values may then be multiplied by the estimated net-benefit bounds to compute a final net cost saving bound, which may serve as a basis for the final ranking of subjects.

The cost prediction model may employ various machine learning (ML) techniques to improve accuracy and adaptability. It may utilize one or more predictive models, including regression models, neural networks, Bayesian models, or reinforcement learning-based approaches. These models may be trained on diverse datasets, including historical claims data, patient demographics, clinical histories, and intervention outcomes, to capture complex cost patterns and predict financial impact with greater precision. By integrating these ML-driven predictions into the ranking system, the disclosed techniques enable a more data-driven, cost-aware prioritization of healthcare interventions.

The final ranking of subjects may be determined by incorporating a two-step process that may account for both the upper and lower net-cost saving bounds. First, the system may assign an initial rank to each subject using standard competition ranking applied separately to the upper and lower bounds. Specifically, a first rank for each subject may be computed based on the upper bound using a ranking method (for example, SciPy's ranking function), which assigns the same rank to subjects with identical upper bounds. Similarly, a second rank for each subject may be computed based on the lower bound. Since these ranks may not always align, they may be averaged to generate a single fractional rank value for each subject. The final ranking order may then be determined by sorting subjects based on this averaged rank. Additionally, the system may incorporate a pairwise surrogate model to analyze feature importance.

According to certain embodiments of the present disclosure, the output of the subject ranking system may comprise a ranked list of the subjects. The ranked list may be organized into distinct subsections or columns for all subjects, providing personal health information (PHI), such as their rank label, their name, additional medical details, or in some aspects, the benefit score in percentage in case of an immediate clinical action (e.g., 30%, 5%, etc.).

The ranking system and associated method disclosed herein can be included as modules of a software application that comprises a backend kernel service and a frontend user interface widget. The software application can be easily integrated as an add on in the existing software tools such as PowerChart™, MessageCenter™ etc. The frontend user interface widget may include sections for summarized information of subjects, their healthcare status including trends and patterns in their encounters, risk categories, benefit scores or the various selection criteria. The user may select various filters or criteria using the frontend user interface widget. The backend kernel service may query and filter the records or documents of the subjects from the EHR database. The backend service may also utilize a role-based access control (RBAC) and identity verification system to authenticate and authorize users and their requests. Moreover, in one embodiment, the backend service can be embedded into cloud platforms—Oracle cloud infrastructure (OCI), Microsoft Azure, Amazon Web Service (AWS) etc.—and can be offered as a service in the cloud.

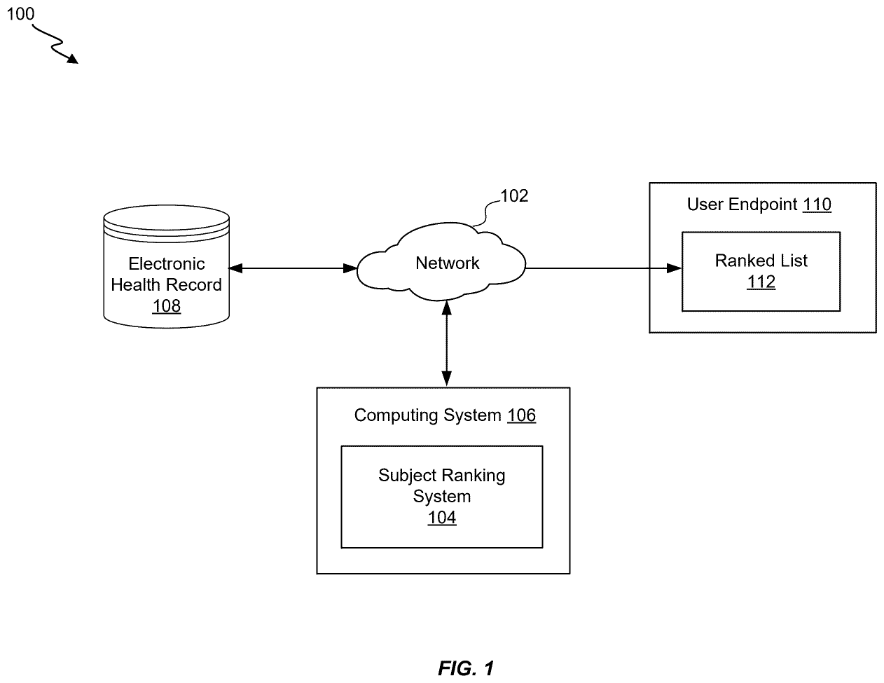

FIG. 1 shows an exemplary system 100 for performing a method to generate a ranked list 112 based on subjects' records in accordance with some aspects of the present disclosure. Exemplary system 100 comprises a computing system 106, a subject ranking system 104, an electronic health record (EHR) 108 or a database, a user endpoint 110, and ranked list 112. The user endpoint 110 may include a tablet, a laptop, a desktop computer, a computer server, and the like. The user endpoint 110 may run an application, a web-based application, or a cloud-app and may provide an interface to the user on the user endpoint 110 for a better user experience. The interface may represent the application authorized and registered to use within a specific territory or may have limited access to other registered individuals. In some instances, the user interface may be a dedicated application with a custom designed graphical user interface (GUI), for example, PowerChart™ or MessageCenter™ application.

The EHR 108 may store a plurality of records of one or more subjects and may be comprised of one or more data storage devices across one or more computers and/or servers. Moreover, the EHR 108 system may comprise one or a plurality of EHR systems such as hospital EHR systems, health information exchange EHR systems, clinical genetics/genomics systems, ambulatory clinic EHR systems, psychiatry or neurology EHR systems, insurance authorizations, and one or more insurance bills generated for interventions performed by the user. In some instances, the EHR 108 may include one or more data stores of health-related records and may further include one or more computers or servers that facilitate the storing and retrieval of the records. In some instances, the EHR 108 or the database may be implemented as a cloud-based platform or may be distributed across multiple physical locations. The EHR 108 may further comprise of systems that can store real-time or near real-time information or data of the subject, for example, data from wearable sensors, bedside monitors, or in-home patient monitors or sensors. In some other instances, the EHR 108 may encompass multiple data storage units distributed across a network of interconnected computers and servers for better scalability, fault tolerance, and efficient data retrieval.

To generate an accurate ranked list 112, the computing system 106 processes subject data using the subject ranking system 104, which prioritizes subjects based on their likelihood to benefit from the predefined intervention (i.e., scheduling a follow-up appointment). The ranked list 112 may then be displayed on the user endpoint 110 for the caregiver or clinician to review. The computing system 106 may be a server or a cloud-based platform providing virtualized resources, for example, OCI, AWS, Microsoft Azure, and Google cloud. In some aspects, the subject ranking system 104 may reside on the user endpoint 110 or the computing device of the user such as laptop, smartphone and the like. Additionally, the subject ranking system 104 and the user endpoint 110 may be communicatively coupled to the EHR 108 through the network 102.

The network 102 may comprise of any form of communication network including public, private, internet, switch, routers, firewalls, and/or similar networks facilitating collaboration, information flow, and seamless connectivity between end nodes. In some embodiments, the network 102 may be a collection of interconnected devices, such as computers, servers, and routers, communicating with each other, enabling data exchange and resource sharing. In other embodiments of present disclosure, the network 102 may be a local area network (LAN) covering a small geographical area with high data transfer rates using ethernet cables or Wi-Fi. The network 102 may be a wide area network (WAN) covering extensive geographical distances and connecting multiple LANs together and/or may include a metropolitan area network (MAN) connecting multiple LANs within a specific organization territory such as hospitals, offices and the like. The other forms of the network 102 with reference to the present disclosure may include any campus area network (CAN), storage area network (SAN) and/or a virtual private network (VPN) to create a secure encrypted connection over a public network (usually the internet). Moreover, the selection of network 102 may depend on factors like scalability, security, and performance requirements.

In some aspects, the intervention can encompass various actions initiated by the user such as see patient, prescription renewal, order lab investigations, diagnostic procedures or dosage adjustments of medications etc. The subject ranking system 104 may be employed to rank the subjects corresponding to the intervention, based on a combination of their benefit bounds and cost savings by leveraging multiple methods and machine learning models, performing a sophisticated analysis of both the health risks without the intervention and the potential benefits of the intervention for each subject (also referred herein as patient). As a result, ranked list 112 may be generated to provide a structured and sortable table or list displaying the rank of subjects with the most net-benefit (i.e., those with high health risk without intervention and greater likelihood of the intervention helping) appear at the top of the list for immediate attention. The ranked list 112 may be presented to the user on the user endpoint 110.

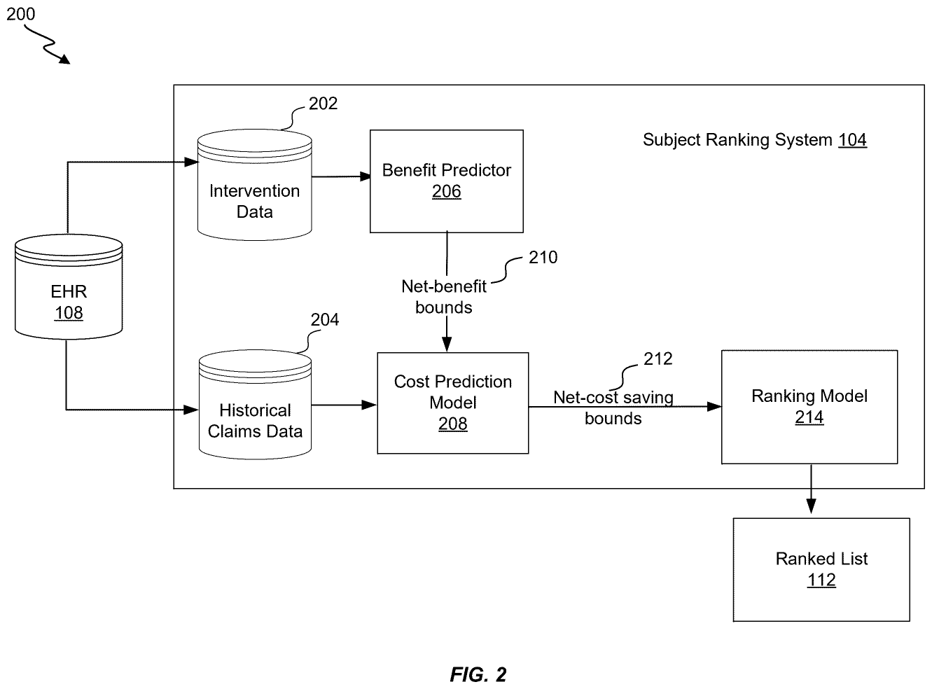

FIG. 2 shows a block diagram of an example overview of the subject ranking system 104 to generate the ranked list 112 in accordance with some aspects of the disclosure. The subject ranking system 104 may include a benefit predictor 206, a cost prediction model 208, and a ranking model 214. The subject ranking system 104 retrieves subject data from the electronic health records (EHR) 108, accessing a wide range of historical and real-time information. This data includes demographic details, clinical history, historical claims data, laboratory results, medication adherence, physician notes, and other relevant medical records. To facilitate structured data access, the system organizes this information within the intervention data 202 and the historical claims data 204 repositories. The intervention data 204 may include various subject specific attributes, confounding features Z, and other contextual data required for applying causal inference techniques.

The intervention data 204 may then be accessed by the benefit predictor 206 that may be utilized to estimate a net-benefit bounds 210 bounds or bounds on the probability of necessity and sufficiency (PNS) of a proposed intervention for each subject. The net-benefit bounds 210 or the upper and lower PNS bounds may indicate the likelihood of positive outcome in the health condition of a subject or avoiding an adverse outcome if the intervention is administered. Machine-learning (ML) models utilized in the benefit predictor 206 may use several underlying mathematical models. For example, the models may be causal inference models designed to estimate net-benefit bounds. These models leverage counterfactual reasoning to predict the expected benefit of an intervention for each subject. The causal inference models may employ techniques such as meta-learning, inverse probability weighting, doubly robust estimation, or other statistical methods to ensure accurate and unbiased effect estimation.

Additionally, the cost prediction model 208 may be configured to calculate a net-cost saving bounds 212 for the predefined intervention, that may quantify the expected benefit of the intervention for each patient by combining clinical data with operational and financial considerations. By doing so, the subject ranking system 104 may rank and prioritize subjects that are most likely to benefit from the intervention while also allocating resources (e.g., hospital staff, funding, and medical bandwidth) in a way that maximizes both clinical impact operational sustainability.

The cost benefit predictor may access historical claims data 204 that may include historical costs of a preventative action (e.g., a cost of a follow-up appointment), historical costs of an acute-care utilization (on an emergency-department visit), and an expected revenue from closing care gaps (performing necessary screenings, vaccinations, or treatments during the appointments). Based on the historical claims data, the system may leverage various machine-learning models to predict the net-cost savings for the predefined intervention.

According to some aspects, the output of the benefit predictor 206 (i.e., the net-benefit bounds 210) may be fed into the cost prediction model 208 to obtain a final net-cost saving bounds 212. This may be achieved by multiplying the predicted cost savings with the net-benefit bounds 210, allowing for a comprehensive measure that accounts for both the effectiveness of the intervention and its financial impact. The net-cost saving bounds 212, that may include upper and lower bounds (C₊ and C₋), quantifies the expected overall value of the intervention for each subject, balancing clinical benefits with economic considerations. This value may serve as a key factor in prioritizing subjects for intervention, enabling a data-driven decision-making process. The net-cost saving bounds 212 may be sent to the ranking model 214 to generate a final ranked list 112.

Finally, the ranking model 214 may perform all functions related to ranking subjects based on their net-cost saving bounds 212, which may include both the upper bound (C+) and the lower bound (C−). The model may process these bounds to assign a final rank score to each subject, ultimately generating a final ranked list 112. This process may involve assigning a first rank to each subject based on the upper bound of the net-cost saving bounds 212 using a standard competition ranking method, followed by assigning a second rank based on the lower bound. These ranks may then be averaged to generate a combined rank score for each subject, and the subjects may be sorted based on this score to produce the final ranked list 112. Additionally, the ranking model 214 may incorporate a pairwise surrogate model to refine ranking decisions, analyze feature importance, and enhance the reliability of the ranking outputs.

The output of the subject ranking system 104, (i.e., the ranked list 112) may be displayed via the user interface. The user endpoint 110 can be a part or subset of the user interface or can be a separate interface (e.g., another GUI page to display the results). In some embodiments, the ranked list 112 may contain various columns or sections, providing detailed information for each subject, such as their rank, name, health condition, or other details.

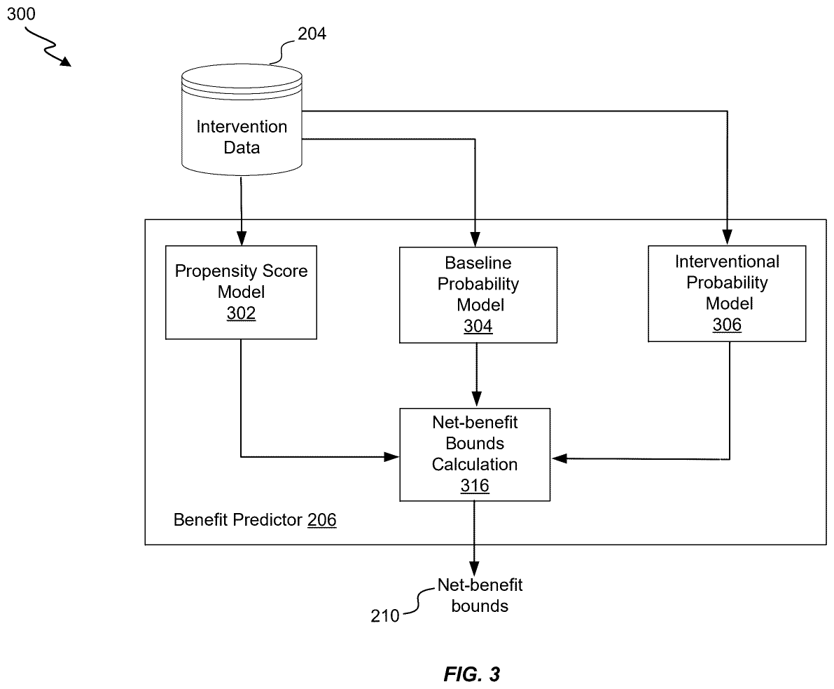

FIG. 3 shows an exemplary block diagram of the benefit predictor 206 for estimating the net-benefit bounds 210 in accordance with some embodiments of the present disclosure. The benefit predictor 206 comprises of a propensity score model 302, a baseline probability model 304, an interventional probability model 306 and a net-benefit bounds calculation 316. Based on various inputs retrieved from both the EHR 108 and the intervention data 204, the benefit predictor 206 may estimate the potential impact of the intervention on each subject, determining the probability of a positive outcome of the intervention on each individual subject.

The benefit predictor 206 may employ three distinct machine-learning models: the propensity score model 302, the baseline probability model 304, and the interventional probability model 306. The propensity score model 302 may predict the likelihood that a subject would receive the intervention under standard care procedures based on their historical data from the EHR 108. This probability, also known as the propensity score, helps quantify the subject's tendency to receive treatment without external influence. Meanwhile, the baseline probability model 304 predicts the expected outcome if the subject does not receive the intervention, and the interventional probability model 306 predicts the subject's expected outcome if the intervention is applied. By comparing these two probabilities with the propensity score, the system determines the net-benefit of the intervention, which may later be used to rank subjects in terms of who would benefit the most. The propensity score model 302 may be designed to predict the probability of the likelihood that a particular subject would receive the intervention under standard care procedures, based on the subject's data from the EHR 108, generating subsequently a propensity score or probability.

The propensity score is a standard component in various types of causal-inference models, as such scores may enable the system to estimate counterfactual outcomes-what would have happened if a subject had (or had not) received the intervention. These models may address confounding variables, which are factors that simultaneously influence both the intervention and the outcome, by estimating the likelihood of treatment solely based on observed data.

The training data used for the causal inference models may be derived from a combination of EHR 108 data and intervention data 204. During training, these models may learn the underlying relationships between confounding features Z, the subject's current health status, associated risk factors, and past interventions. By capturing these complex patterns, the causal models may accurately predict the probability, or propensity, that a similar subject would receive the intervention under standard care procedures. Since the likelihood of an intervention is influenced by the subject's health condition and risk profile, the propensity score model 302 may account for these factors to generate reliable estimates. The propensity score or probability may be represented in mathematical terms by P(X|Z), where P is the probability of the intervention X happening given features Z. The output probability serves as an input for further analysis in determining the overall benefit of the intervention, contributing to the final net-benefit bounds 210.

Simultaneously, the baseline model 304 may generate a baseline probability represents the likelihood of achieving a positive outcome without the intervention often represented as: P(Y_{X=0}=1), wherein P is the probability of a specific outcome associated with the intervention, X is the intervention and Y is the outcome. In contrast, the interventional model 306 may generate an interventional probability that estimates the likelihood of achieving the positive outcome with the intervention represented as: P(Y_{X=1}=1). Both the baseline probability model 403 and the interventional probability model 306 may use causal inference models to assess the effect of the intervention by predicting outcomes both with and without the intervention. In this way, it is somewhat related to an A/B test, predicting separately what would happen under both conditions A and B. However, unlike a traditional A/B test which requires population-level randomization, this approach predicts both the interventional and baseline probabilities for each individual subject. Collectively, the propensity score model 302, the baseline probability model 403 and the interventional probability model 306 may be referred in the disclosure throughout as “causal inference models”.

Once the propensity score, baseline probability, interventional probability, have been calculated using the causal inference models, these outputs may be passed to the net-benefit bounds calculation 316 that may aggregate the probabilities and adjusts for confounding factors, ultimately generating a net-benefit bounds 318 (also referred herein as the probability of necessity and sufficiency (PNS) or PNS bounds) for each subject. The equation (Eq. 1) below is used to compute the subject's risk probability, given the output of the three causal models, where P(Y|Z) is the probability of a positive outcome given the subject's state.

$$P ( Y \vert Z ) = P ( {Y}_{X = 1} = 1 ) P ( X \vert Z ) + P ( {Y}_{X = 0} ) - P ( {Y}_{X = 0} = 1 ) P ( X \vert Z ) , \qquad Eq . 1$$

where P(Y|Z) represents the probability of a positive outcome Y given confounding features Z.

According to some aspects of the present disclosure, subject's data may include any data relevant to the subject that may affect the outcomes for the subject. As there may be a vast number of potential factors affecting outcomes, subject's data may often be incomplete. Resultantly, probabilities estimated derived from the subject's data may not exactly describe the subject. This difference in the calculated probability may be accounted for by instead calculating the net benefit score as an upper and lower PNS bounds. The upper and lower PNS bounds may be defined in accordance with Pearl, J. (2009). Causality. Cambridge University Press (which is hereby incorporated by reference in its entirety for all purposes) and/or as:

### Lower Bounds:

$$max \{ \begin{matrix}0 \\ P ( {Y}_{X = 1} = 1 ) - P ( {Y}_{X = 0} = 1 ) \\ P ( Y = 1 ) - P ( {Y}_{X = 0} = 1 ) \\ P ( {Y}_{X = 1} = 1 ) - P ( Y = 1 ) \\ - x , x < 0 \\ x , x \geq 0\end{matrix} \} \leq PNS , \qquad Eq . 2$$

### Upper Bounds:

$$P N S \leq min \{ \begin{matrix}P ( {Y}_{X = 1} = 1 ) \\ P ( {Y}_{X = 1} = 0 ) \\ P ( X = 1 , Y = 1 ) + P ( X = 0 , Y = 0 ) \\ P ( {Y}_{X = 1} = 1 ) - P ( {Y}_{X = 0} = 1 ) + P ( X = 1 , Y = 0 ) + P ( X = 1 , Y = 0 )\end{matrix} \} , \qquad Eq . 3$$

where P(Y_{X=1}=1) is the probability that outcome Y would be 1 (i.e., a positive outcome) if intervention X occurs (i.e., X=1), P(Y_{X=0}=0) is the probability that outcome Y would be 0 (i.e., a negative outcome) if intervention X does not occur (i.e., X=0),), and P(Y_{X=0}=1) is the probability that outcome Y would be 1 (i.e., a positive outcome) if intervention X does not occur (i.e., X=0). Similarly, P(Y_{X=1}=1) P(X|Z) and P(Y_{X=0}=1) P(X|Z) are weighted probabilities, combining counterfactual probabilities with the likelihood of X given Z. Finally, P(Y=1) is defined in accordance with the output from Eq. 1. Thus, Eq. 1 facilitates setting bounds in accordance with Eqs. 2-3 in a practical manner. Furthermore, to address the overdetermined nature of the terms involved in Eq. 2, Eq. 1 may serve as a normalization constraint that allows P(Y|Z) to be computed from the outputs of the other three trained models (i.e., the causal inference models). This avoids the need to model each component of Eq. 2 separately and ensures internal consistency across probability estimates.

To train the ML models, the benefit predictor 206 may utilize intervention data 204 obtained from the EHR 108, including medical conditions, lab results, prior procedures, medications, and social determinants of health. The data may be divided into groups where the interventions were applied and where they were not, allowing for the construction of the ML models that accurately reflect real-world scenarios. Additional steps, such as double-stratified sampling, may be used to balance the data and prevent the causal models from being biased by the asymmetric mixture in the training data, of individuals receiving the intervention or not and having a positive or negative outcome.

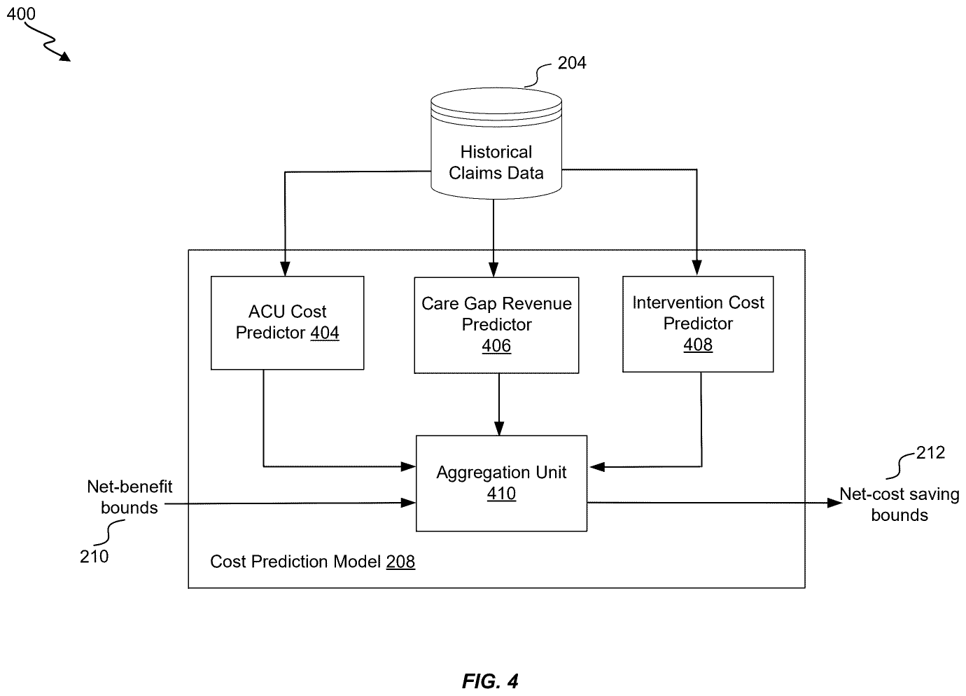

FIG. 4 shows an exemplary block diagram of the cost prediction model 208 to predict net-benefit cost savings 212 in accordance with some embodiments of the present disclosure. The cost prediction model 208 may include an ACU cost predictor 406, a care gap revenue predictor 406, an intervention cost predictor 408, and an aggregation unit 410. Each of these components plays a crucial role in estimating potential cost savings by analyzing historical claims data 204 retrieved from the EHR 108. The cost prediction model 208 evaluates the financial impact of an intervention by computing expected costs under both intervention and non-intervention scenarios, ensuring an informed decision-making process for ranking subjects based on economic and clinical benefit.

The cost prediction model 208 may access the historical claims data 204 of each subject from the EHR 108 to estimate the cost savings. The ACU cost predictor 406 may estimate the cost associated with acute-care utilization (ACU) if the subject's outcome is negative. This estimation relies on the historical ACU costs for similar subjects, adjusted for individual risk factors such as disease severity, prior hospitalization history, and comorbidities. The likelihood of ACU occurrence without intervention may be used to predict the expected cost in cases where the subject does not undergo the intervention. Similarly, the intervention cost predictor 408 estimates the cost to see the subject during the intervention, represented by “R”, which may include direct expenses such as medical procedures, physician consultations, and diagnostic tests. The intervention cost predictor 408 may leverage historical claims data 204 to identify subjects with similar medical conditions and predict the associated intervention costs.

Furthermore, the care gap revenue predictor 406 may be used to estimate the expected revenue incurred for closing care gaps as a result of the intervention or during the intervention, represented as “G”. Care gaps refer to missed medical services or preventive measures that, if addressed, may improve patient outcomes and reduce future healthcare costs. The model analyzes historical reimbursement patterns, adherence to care guidelines, and provider incentives to predict potential revenue gains. By integrating this revenue estimation, the care gap revenue predictor 406 enables the system to prioritize interventions based on both financial sustainability and clinical efficacy. To calculate the overall cost savings, the aggregation unit 410 may combine predictions from the ACU cost predictor 404, intervention cost predictor 408, and care gap revenue predictor 406.

Finally, to compute the net-cost saving bounds 212, the aggregation unit 410 may integrate net-benefit bounds 210 with the predicted cost savings. The net-benefit bounds 210 are derived from the benefit predictor 206, which estimates the clinical impact of an intervention using causal inference models. The final net-benefit cost savings may be computed as:

$$Net - benefit cost savings = PNS * ( A + G - R ) , \qquad Eq . 5$$

where PNS represents the predicted net-benefit bounds 210, quantifying the clinical value of the intervention. Since the net-benefit bounds 210 includes two values of PNS per patient, (i.e., PNSᵤₚₚₑᵣ, and PNS_{tower}) the net-cost saving bounds 212 may also be evaluated for each end of the bounds separately. This may result in upper and lower bounds on the net-benefit cost savings, too, denoted by C₊ and C₋. The final ranking of the subjects may be based upon each subjects' values for C₊ and C₋. The net-cost saving bounds 212 may further be processed by the ranking model 214 to compute the final ranked list 112.

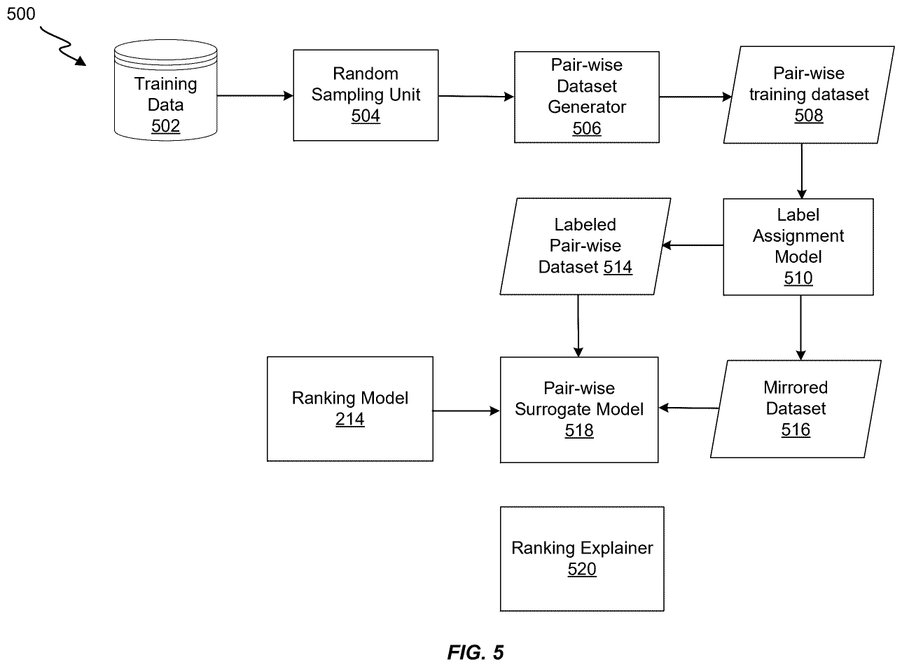

FIG. 5 shows a training process of the pair-wise surrogate model 214 for analyzing feature importance of subjects during ranking in accordance with some aspects of the present disclosure. The training process may include training data 502, a random sampling unit 504, a pair-wise dataset generator 506, a label assignment model, the ranking model 214, and the pair-wise surrogate model 214. The pairwise surrogate model 518 may play a complementary role to the ranking model 214 by helping to analyze, interpret and explain its ranking decisions. The output of the ranking models 214 may be used by the pairwise surrogate model 518 during training, which may then become a ground truth for training the surrogate model 518. The pairwise surrogate model 518 may be trained as a binary classifier to predict whether a subject A should be ranked higher than a subject B. It may use cross-entropy loss, or similar loss functions (e.g., hinge loss or pairwise logistic loss), to compare its predicted probability with the actual ranking decision made by the ranking model 214. If the prediction of the pairwise surrogate model 518 turns out to be incorrect, the cross-entropy loss would be high, pushing the model to adjust its parameters.

The training process may begin by creating training examples where each instance may consist of two patients' feature vectors concatenated together. The training data 502, which may comprise of individual patient records and their associated features, may be accessed by the random sampling unit 504. Since the data may include n patients, there may be (n²−n) possible unique patient pairs (excluding self-comparisons). Training on all these pairs may be computationally infeasible due to the sheer volume of data. To address this challenge, the random sampling unit 504 may select a random subset of patient pairs, ensuring a balance between computational efficiency and dataset diversity. This random selection process may prevent overfitting to specific patient comparisons and allows the pair-wise surrogate model 518 to generalize well to unseen data. The randomly selected patient pairs may then be sent to the pair-wise dataset generator 506, where their respective feature vectors may be extracted and prepared for further processing.

By leveraging the pair-wise dataset generator 506, the feature vectors of each randomly selected patient pair may be concatenated to form a single feature representation. Each patient may initially be characterized by a high-dimensional feature vector containing approximately many (e.g., thousands of) features. By concatenating the feature vectors of two patients in a pair, the resulting pair-wise feature representation may have a dimensionality that is multiple times higher than the number of features. The concatenation process may be important because the pair-wise surrogate model 214 need to be trained as a binary classifier, i.e., it may not evaluate individual patients directly but instead compares them in pairs to determine which one should be ranked higher. The pair-wise dataset generator 506 allows the training samples to be structured appropriately for the comparison-based learning approach.

Once the pair-wise training dataset 508 may be generated, it may be passed into the label assignment model 510. The label assignment model 510 assigns binary ranking labels to each pair by comparing their existing net-benefit estimates (which may represent their net-benefit cost savings). For example, if the subject A has a higher net-benefit estimate than the subject B, the pair may be assigned a label of 1 (indicating subject A should be ranked higher). Conversely, if subject B has a net-benefit estimate than subject A, the label is 0. The output of the label assignment model 510 may be a labeled pair-wise dataset 514, which now comprises feature vectors along with their respective ranking labels.

To ensure that the pair-wise surrogate model learns ranking order correctly, the label assignment model 510 may also generate a mirrored dataset 516. This may be done by swapping the feature positions of the patient pairs (i.e., switching subject A and subject B) within each pair and flipping the corresponding label. For instance, if the original pair (subject A, subject B) was labeled 1, the flipped pair (subject B, subject A) will be labeled 0. Both the labeled pair-wise dataset 514 and the mirrored dataset 516 may be used to train the pair-wise surrogate model, so that model may learn to rank patients correctly regardless of feature position. This may enable the pairwise surrogate model 518 to learn from relative ranking decisions rather than absolute ranking scores.

Finally, both the labeled pair-wise dataset 514 and the mirrored dataset 516 may be fed into the pair-wise surrogate model 518 for training. The pairwise surrogate model 518 (trained as a binary classifier) may then learn to predict whether one patient ranks higher than another using a loss function, such as a cross-entropy loss, or another suitable loss function (e.g., hinge loss, pairwise logistic loss, etc.), by comparing its predicted output with the actual ranking made by the ranking model 214. Thus, since the ranking model 214 generates a net-benefit-based ranking, the pairwise surrogate model 518 (binary classifier) may learn patterns in how the ranking model 214 differentiates subjects based on their features. Moreover, the inclusion of both datasets (the labeled pair-wise dataset 514 and the mirrored dataset 516) effectively doubles the number of training samples, improving model robustness and generalization. Through training, the pairwise surrogate model 518 may learn to approximate the behavior of the ranking model 214 based on pairwise comparisons.

Once trained, the pairwise surrogate model 518 may enable interpretation of the final rankings during inference by analyzing how feature changes affect rankings. To generate explanations, each individual subject may be compared to a group of comparison patients from a different ranking tier. Using SHAP (Shapley Additive Explanations) or similar techniques, the pairwise surrogate model 518 may compute feature contributions for each comparison, and these individual explanations may then be aggregated to produce a composite explanation for why a given subject is ranked within a particular tier. This approach provides a nuanced, group-relative interpretation of ranking decisions, showing how specific features influence a subject's position relative to others. By leveraging these aggregated insights, the system improves transparency and supports refinement of both the ranking model 214 and the underlying machine-learning methodology.

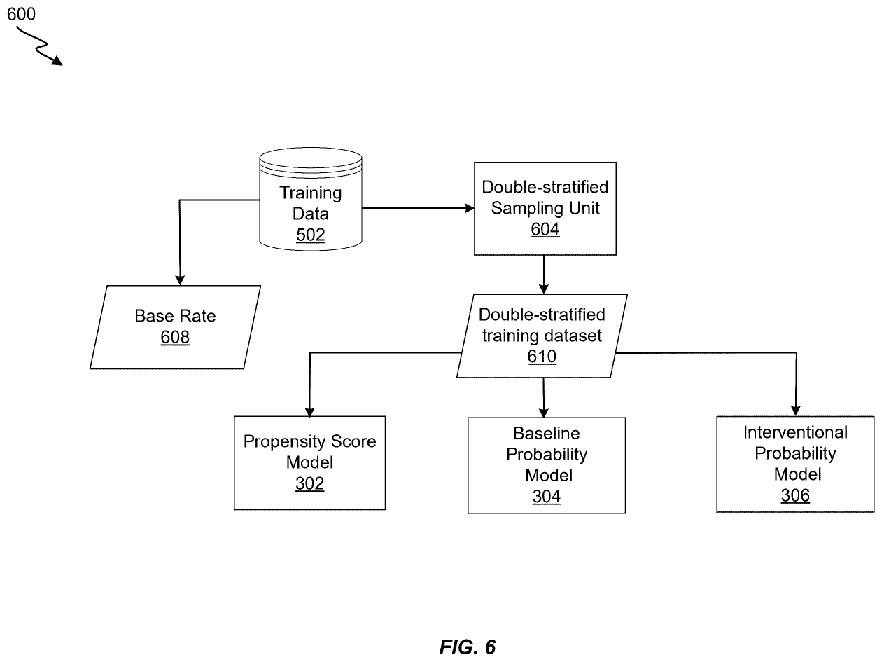

FIG. 6 shows a training process of the causal inference models for predicting the net-benefit bounds 210 by using a double-stratified sampling technique. The training process may include training data 502, a double-stratified sampling unit 604, a double-stratified training dataset 610, a base rate 608, and the three causal inference models (i.e., the propensity score model 302, the baseline probability model 304, and the interventional probability model 306).

Training data 502 may be obtained from the EHR 108 to train the causal inference models but in order to practically work with large datasets, the EHR data may need to be subsampled first. For this purpose, stratification sampling technique may be used to capture the training data that may provide sufficient coverage across exposures and outcomes so that each of the models (counterfactual models and propensity score models) may achieve reasonable performance.

The occurrence rates of the data-slices for X={0,1}, Y={0, 1} may be substantially different. For an amount of data that may be used for training, there may be few (X=0, Y=0) samples and relatively greater number of (X=1, Y=1) samples, for example. Similarly, there may be a large number of confounder features (for example, of order 10\hat{ }4 confounding features, collectively represented by “Z”, the vector of confounding features), which makes the training-data size even larger. To take samples from the full data in a non-stratified way, there would be a need for an infeasible amount of computer storage in order to support training of the rarer X, Y combinations at an acceptable performance level. Instead, the system may re-sample from X and Y in a biased way so that there may be an equal number across each of the 4 combinations. Such an approach may be referred as “double-stratified sampling”.

The double-stratified sampling unit 604 may be used to systematically re-balance the training data 502 by equally representing each of the four (X, Y) combinations e.g., (X=0, Y=0), (X=0, Y=1), (X=1, Y=0), and (X=1, Y=1). This may be achieved by under-sampling the over-represented combinations, effectively addressing data imbalance without requiring excessive storage or computation. The output of the double-stratified sampling unit may be a double-stratified training dataset 610, on which the causal inference models may be trained on. Furthermore, the double-stratified sampling unit 604 converts the training data 502 into a manageable size while maintaining sufficient representation of all scenarios. However, such an approach of subsampling may disrupt the probability prediction of the causal inference models by skewing the training distribution relative to the full-data distribution. Fortunately, the subject ranking system 104 may repair the induced bias after training so that the model outputs probabilities can be calibrated to the full-data distribution. This may be achieved by adjusting the output probabilities of the causal models using a known base rate 608 probability in the training data 502. It may computationally be advantageous to obtain the base rate in the training data 502 by counting the fraction of each X={0,1} and Y={0,1} occurrence. The counting operation may provide with a value for the base rate, without any training or other effort. Then the system may train on the subsampled, double-stratified training dataset 610 with an equal mix of rare and non-rare classes so that the causal inference models may learn from data with good coverage over the rare-class feature-behavior. Finally, at inference, the system may adjust the output probabilities from the “biased” causal inference models using the known base rate 608 from the training data 502. The base rate may refer to the overall frequency or proportion of an event occurring in a dataset. For example, it may represent how often a particular outcome (e.g., a patient responding to treatment) occurs in the training data 502 versus the full dataset (the intervention data or the EHR 108 data).

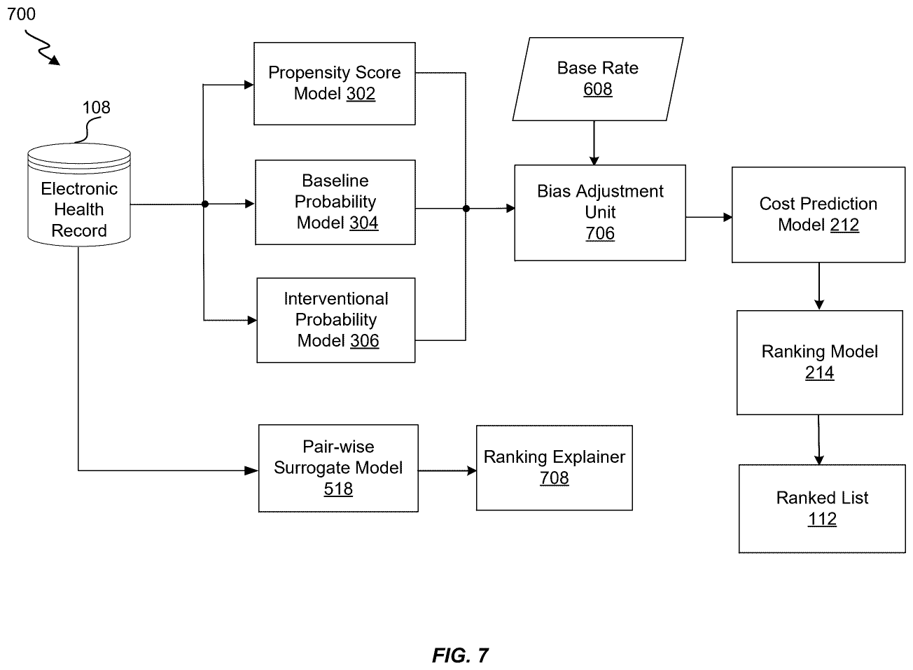

FIG. 7 illustrates an exemplary overview of the subject ranking system 104 at inference, incorporating a bias adjustment unit 706 to calibrate output of the causal model in accordance with some embodiments of the present disclosure. Since the data upon which the causal inference models are trained (double-stratified training dataset 610) may be biased due to double-stratified sampling (which balances the occurrence of different (X, Y) pairs), the base rate 608 “b” in the training data 502 may differ from the true base rate b′ in the full dataset (the real world data at inference). The output of each of the three causal models may be adjusted using the bias adjustment unit 706. The bias adjustment unit 706 may take the base rate 608 in the training data 502 to calibrate the outputs to a full-data domain. The formula employed by the system for bias adjustment of the output of the causal-inference models is:

$${p}^{′} = {b}^{′} \frac{p - p b}{b - p b + {b}^{′} p - {bb}^{′}} , \qquad Eq . 5$$

where p′ is the adjusted probability output by the causal inference models after bias correction, b is the base-rate in the training data 502, b′ is the base-rate in the full data at inference, and p and is the probability predicted by the causal inference models before bias adjustment.

This adjustment may be applied to each of the three causal inference models, i.e., the propensity score model 302, the baseline probability model 304, and the interventional probability model 306. This adjustment is performed independently of the computation of the net-benefit bounds 210. The calibrated outputs from the bias adjustment unit 706 may then serve as inputs to the formula used to compute the net-benefit bounds 210 (via the cost prediction model 212). These bounds may subsequently be used by the ranking model 214 to generate the final ranked list 112. The trained pairwise surrogate model 518 may assist in interpreting and validating the decisions of the ranking model 214 by analyzing feature contributions and providing transparency in ranking outcomes.

During inference, the ranking model 214 generates final ranked list 112 using net-cost saving bounds 212 (C+ and C−) and the described ranking methodology. The pairwise surrogate model 518 may be used for post-hoc interpretability by comparing a patient's ranking against a reference group. A target patient may be compared to various groups, such as median-ranked patients, high-risk groups, or randomly selected patients, to assess ranking consistency and significance.

The pairwise surrogate model 518 predicts the pairwise rank for each comparison by estimating whether the target patient would be ranked higher or lower relative to each reference patient. This allows for an in-depth understanding of ranking behavior and helps identify which confounder features are substantial, significant or most significant in determining the rank assigned to the subject. Outputs from the trained pairwise surrogate model 518 can (optionally) be output to a ranking explainer 708, that may generate natural language summaries, visual interfaces, or other user-facing content, etc., on the user endpoint 110 that can identify some or all of the rank data. Optionally, the ranking explainer may also provide logic or a rationale for such rankings and/or explanations.

Finally, the generated ranked list 112 may display patients in descending order of expected benefit, enabling the caregiver or user to prioritize those with the highest predicted impact for treatment or resource allocation.

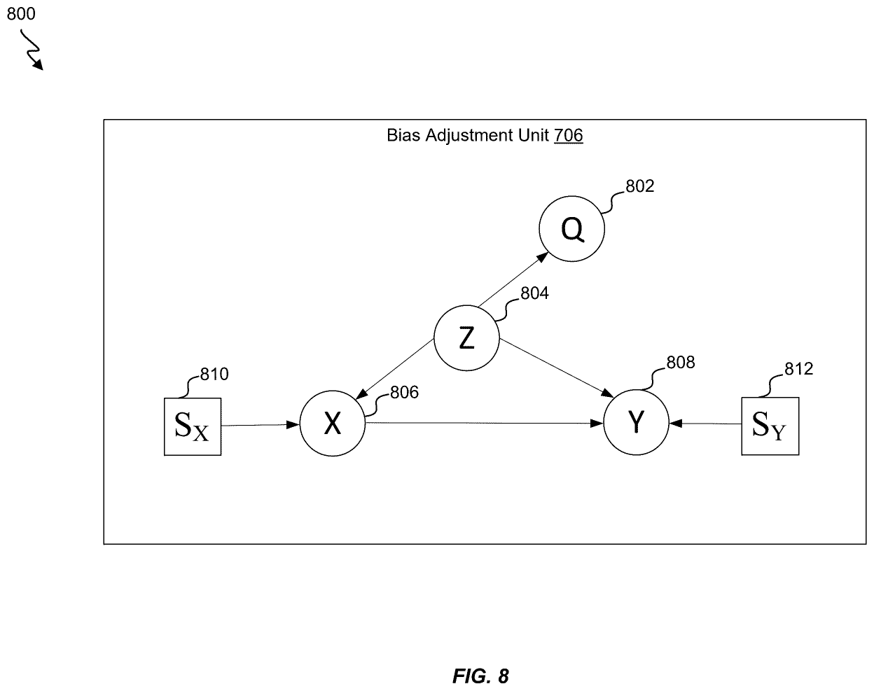

FIG. 8 shows a selection diagram within the bias adjustment unit 706 detailing the transportability process from a double-stratified training domain to a full-data (real-world) domain for probability estimates. The adjustment addresses bias introduced by stratifying on both intervention assignment X 806 and outcome Y 808 when these are confounded by an external factor Z 804. Given that the training data has been double-stratified, an adjustment formula may be necessary to correctly transport these probabilities to the real-world setting and achieve proper calibration. To achieve this, a joint probability distribution in the full-data domain is expanded using a chain rule, incorporating selection variables Sₓ 810 and S_{y} 812 which may denote the double-stratification process explained in FIG. 6 (double-stratified sampling unit 604). Moreover, Sₓ 810 and S_{y} 812 indicate that the data has been selected (stratified) based on the values of X 806 and Y 808 before being used for model training. To further refine the calibration, the selection diagram introduces a new variable Q 802, representing the causal inference model's predicted probability score at inference time. The definition of how to transport between domains is formally given by the relation:

$$P ( Q , Y , X ) = P ( Q , Y , X \vert {S}_{Y} , {S}_{X} ) , \qquad Eq . 6$$

Equation 6 expresses that the real-world probability distribution of the model's predicted probability score Q 802, the outcome Y 808, and the treatment X 806 can be derived from the training domain distribution, which was influenced by the selection variables (S_{y}, Sₓ). In other words, it shows how to “transport” probability estimates from a biased, stratified training dataset to an unbiased, real-world dataset by accounting for the selection process during training. Additionally, when transporting data to a different domain by stratification, the action being stratified may be mathematically represented as conditioning on either or both if double-stratification is being applied.

The joint probability distribution may be calculated using:

$$P ( Q , Y , X \vert {S}_{Y} , {S}_{X} ) = P ( Q \vert Y , X , {S}_{Y} , {S}_{X} ) P ( Y \vert X , {S}_{Y} , {S}_{X} ) P ( X \vert {S}_{Y} , {S}_{X} ) \qquad Eq . 7$$

Equation. 8 reflects a high-level result derived from applying Bayes' rule to adjust for dataset shift between the development (biased) sample and the full-data domain. It begins with the chain rule of probability, which breaks down the joint distribution into three components: P(Q|Y, X, S_{Y}, S_{X}) (the probability of the model's predicted score Q 802 given the outcome Y 808, treatment X 806, and selection variables S_{Y} 812, S_{X} 810), P(Y|X, S_{Y}, S_{X}) (the probability of the outcome Y given the treatment X and the selection variables), and P(X|S_{Y}, S_{X}) (the probability of the treatment assignment X given the selection process S_{Y} 812, S_{X} 810).

The goal of the bias adjustment unit 606 may be to calibrate the probability outputs of the trained causal inference models by introducing an additional logistic regression model. This model may be trained on the original model outputs and provides recalibrated probabilities in the real-data domain. The final bias adjustment formula for the transported probability of Y=1 given Q and X may be derived as:

$${P}^{′} ( Y = 1 \vert Q , X ) = \frac{P ( Y = 1 \vert Q , X ) {P}^{′} ( Y = 1 \vert X )}{\begin{matrix}P ( Y = 1 \vert Q , X ) {P}^{′} ( Y = 1 \vert X ) + \\ P ( Y = 1 \vert X ) ( 1 - P ( Y = 1 \vert Q , X ) )\end{matrix}} \times \frac{1 - P ( Y = 1 \vert X )}{1 - P ( Y = 1 \vert X )} , \qquad Eq . 8$$

The Eq. 8 may enable the causal inference model predictions to remain valid after they are transported to the full-data domain, aligning with the real-world base-rate distributions of the patient population. Additionally, the base rates used in the adjustment process may be computed separately for the treatment and control groups.

The final form of the transportability equation may align with Theorem 2 of Elkan (2001) “The Foundations of Cost-Sensitive Learning”, which is hereby incorporated by reference in its entirety for all purposes. This connection helps validate that, even in the presence of confounding, the bias correction formula remains consistent with traditional rescaling techniques. Specifically, the adjustment is given by:

$${P}^{′} ( Y = 1 \vert Q , X ) = \frac{Q - Q B}{B - Q B + {B}^{′} Q - {BB}^{′}} , \qquad Eq . 9$$

This expression corresponds to Eq. 5 employed by the bias adjustment unit 606 for recalibrating the outputs of the causal inference models, as illustrated by FIG. 6. By leveraging this form, the bias adjustment unit 606 may apply principled post-hoc correction to the predicted probabilities, allowing models trained on stratified or biased data to provide estimates that more accurately reflect the distribution of outcomes in real-world patient populations.

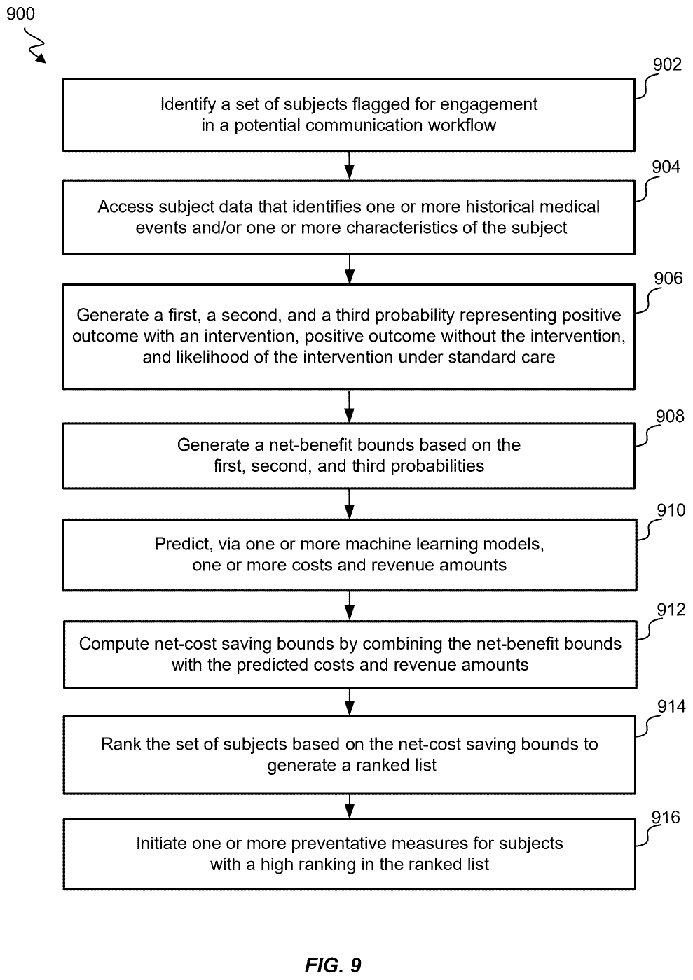

FIG. 9 shows an example flowchart of a system for generating intervention success probabilities and ranking the subjects in accordance with some embodiments of the present disclosure. At block 902, a set of subjects may be flagged for engagement in a potential communication workflow that may include consultations for treatment or one or more clinical actions to be conducted by the user or the clinician. The subject data associated with the set of subjects may be accessed that identifies one or more historical medical events and/or one or more characteristics of the subject such as demographic and personal details that may include age, gender, health condition, etc., at block 904. The subject data may be further used by the system to predict various metrics associated with each subject of the set of subjects.

At block 906, the first probability that may indicate a positive outcome of the intervention, and the second probability that may indicate a positive outcome without the intervention, and a third probability of a likelihood of the subject receiving the intervention based on their characteristics may be generated. To generate these probabilities, causal inference models may be used which provide a structured way to predict outcomes and evaluate the impact of different scenarios on different subjects. Based on the first, the second, and the third probability, a net-benefit bound may be generated for each subject of the set of subjects, at block 908. The net-benefit bounds may represent a quantified value of a potential improvement in the health status of the patient or the subject because of doing the intervention or clinical action. Additionally, at block 910, one or more costs and revenue amounts may be predicted using a cost prediction model. The one or more costs and revenue amounts may include: a cost associated with performing the intervention, a cost associated with an acute-care utilization or an emergency department visit due to a negative outcome, and a revenue generated from closing care gaps by performing the predefined intervention. The cost prediction model may leverage various ML models to predict the one or more cost and revenue amounts, including regression models, neural networks, Bayesian models, or reinforcement learning-based models.

Finally, at block 912, the net-benefit bounds may be combined with the predicted one or more costs and revenue amounts to compute a net-cost saving bounds. The computed bounds may be used to establish a rank of each subject of the set of subjects to generate a ranked list 112, at block 914. Once the ranked list 112 may be generated, one or more preventative measures may be initiated for subjects a high ranking in the ranked list, at block 916. By applying these techniques, the subject ranking system 104 can construct a comparative picture of the benefits and potential risks associated with the predefined intervention, facilitating in informed decision-making. While the present disclosure focuses on a specific intervention (i.e., scheduling a follow-up appointment), the system may be extended to evaluate various other types of interventions. This may be achieved by training separate net-benefit and cost-savings models for each intervention of interest, allowing the subject ranking system 104 to estimate individualized impact per intervention. Potential interventions may include initiating medication, assigning a case manager, recommending lifestyle programs (e.g., smoking cessation or diabetes prevention), or prioritizing diagnostic screenings. In this way, the disclosed techniques may support flexible and tailored decision-making across a diverse set of clinical or operational interventions.

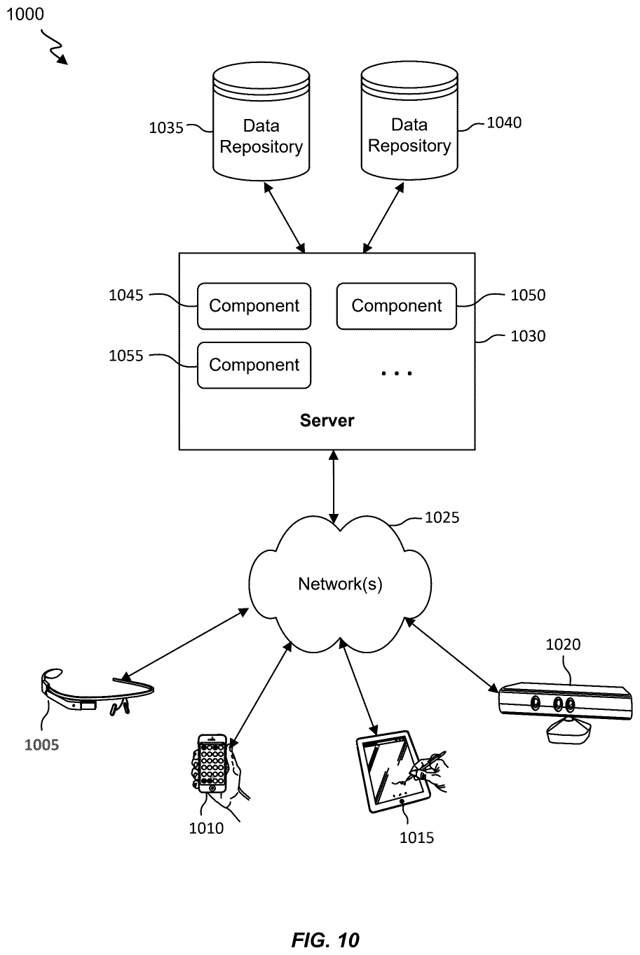

FIG. 10 illustrates a simplified diagram of an example distributed system 1000 for a cloud hosting the subject ranking system 104. In the illustrated example, the distributed system 1000 includes one or more client computing devices 1005, 1010, 1015, and 1020, coupled to a server 1030 via one or more communication networks 1025. The clients computing devices 1005, 1010, 1015, and 1020 may be configured to execute one or more applications interact with the server 1030 to access and utilize the subject platform securely integrated within a cloud environment, such as Oracle cloud integrated with Cohere. Within this framework, the server 1030 is configured to host and manage a range of services or software applications, facilitating seamless integration and operation of the subject management platform.

In various aspects, the server 1030 may extend its capabilities to encompass additional services or software applications. These services may span both virtual and non-virtual environments, enabling a comprehensive and adaptable infrastructure for securely deploying GenAI solutions within the cloud ecosystem. In some respects, these services may be offered as web-based or cloud services, such as under a Software as a Service (SaaS) model to the users of the client computing devices 1005, 1010, 1015, and/or 1020. Users operating the client computing devices 1005, 1010, 1015, and/or 1020 may in turn utilize one or more client applications to interact with the server 1030 to utilize the services provided by these components. Furthermore, client computing devices 1005, 1010, 1015, and/or 1020 may in turn utilize one or more client applications to initiate and manage specific tasks or analyses within the Subject ranking system.

In the configuration depicted in FIG. 10, the server 1030 may include one or more components 1045, 1050 and 1055 that implement the functions performed by the server 1030. These components may include software components that may be executed by one or more processors, hardware components, or combinations thereof. It should be appreciated that various system configurations are possible, which may differ from distributed system 1000. The example shown in FIG. 10 is thus one example of a distributed system for implementing an example system and is not intended to be limiting.

Users may initiate requests for the subject ranking system through client computing devices 1005, 1010, 1015, and/or 1020 for inference or other machine-learning tasks. A client device may provide an interface that enables a user of the client device to interact with the subject ranking system. The client device may also output information to the user via this interface. Although FIG. 10 depicts only four client computing devices, any number of client computing devices may be supported providing scalability and accessibility within the integrated subject ranking system on the cloud.

The client devices may include various types of computing systems, such as portable handheld devices, general purpose computers, such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems, such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone®), tablets (e.g., iPad®), personal digital assistants (PDAs), and the like. Wearable devices may include Google Glass® head mounted display, and other devices. Gaming systems may include various handheld gaming devices, Internet-enabled gaming devices (e.g., a Microsoft Xbox® gaming console with or without a Kinect® gesture input device, Sony PlayStation® system, various gaming systems provided by Nintendo®, and others), and the like. The client devices may be capable of executing various applications, such as various Internet-related apps, communication applications (e.g., E-mail applications, short message service (SMS) applications) and may use various communication protocols.

Network(s) 1025 may be any type of network familiar to those skilled in the art that can support data communications using any of a variety of available protocols, including without limitation TCP/IP (transmission control protocol/internet protocol), SNA (systems network architecture), IPX (internet packet exchange), AppleTalk®, and the like. Merely by way of example, network(s) 1025 can be a local area network (LAN), networks based on ethernet, token-ring, a wide-area network (WAN), the internet, a virtual network, a virtual private network (VPN), an intranet, an extranet, a public switched telephone network (PSTN), an infra-red network, a wireless network (e.g., a network operating under any of the institute of electrical and electronics (IEEE) 1002.10 suite of protocols, Bluetooth®, and/or any other wireless protocol), and/or any combination of these and/or other networks.

The server 1030 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The server 1030 can include one or more virtual machines running virtual operating systems, or other computing architectures involving virtualization, such as one or more flexible pools of logical storage devices that can be virtualized to maintain virtual storage devices for the server. In various aspects, the server 1030 may be adapted to run one or more services or software applications that provide the functionality described in the foregoing disclosure.

The computing systems in the server 1030 may run one or more operating systems including any of those discussed above, as well as any commercially available server operating system. The server 1030 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

Distributed system 1000 may also include one or more data repositories 1035, 1040. Data repositories 1035, 1040 may reside in many locations. For example, a data repository used by the server 1030 may be local to server 1030 or may be remote from the server 1030 and in communication with the server 1030 via a network-based or dedicated connection. Data repositories 1035, 1040 may be of different types. In some instances, a data repository used by the server 1030 may be a database, for example, a relational database, such as databases provided by Oracle Corporation® and other vendors. One or more of these databases may be adapted to enable storage, update, and retrieval of data to and from the database in response to structured query language (SQL)-formatted commands. In some aspects, one or more data repositories 1035, 940 may also be used by applications to store application data. The data repositories used by applications may be of different types, such as, for example, a key-value store repository, an object store repository, or a general storage repository supported by a file system.

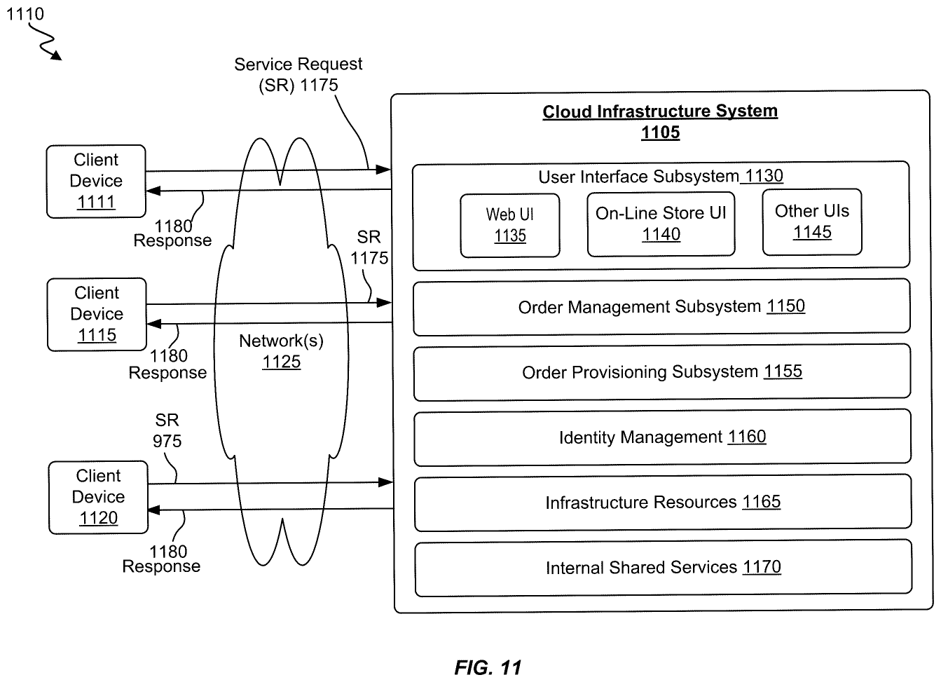

FIG. 11 is a simplified block diagram of a cloud-based system environment in which various services of the server 1030 of FIG. 10 may be offered as cloud services, in accordance with certain aspects. In the illustrative example depicted in FIG. 11, cloud infrastructure system 1105 may provide one or more cloud services that may be requested by users using one or more client devices 1110, 1115, and 1120. Cloud infrastructure system 1105 may comprise one or more computers and/or servers that may include those described for server 1030. The computers in cloud infrastructure system 1105 may be organized as general-purpose computers, specialized server computers, server farms, server clusters, or any other appropriate arrangement and/or combination.

Network(s) 1125 may facilitate communication and exchange of data between client devices 1111, 1115, and 1120 and cloud infrastructure system 1105. Network(s) 1125 may include one or more networks. The networks may be of the same or different types. Network(s) 1125 may support one or more communication protocols, including wired and/or wireless protocols, for facilitating the communications.

The illustrative example depicted in FIG. 11 is only one example of a cloud infrastructure system 1105 and is not intended to be limiting. It should be appreciated that, in some other aspects, cloud infrastructure system 1105 may have more or fewer components than those depicted in FIG. 11, may combine two or more components, or may have a different configuration or arrangement of components. For example, although FIG. 11 depicts three client computing devices, any number of client computing devices may be supported in alternative aspects.

The term cloud service is generally used to refer to a service that is made available to users on demand and via a communication network, such as the internet by systems (e.g., cloud infrastructure system 1105) of a service provider. Typically, in a public cloud environment, servers and systems that make up the cloud service provider's system are different from the client's own on-premises servers and systems. The cloud service provider's systems are managed by the cloud service provider. Clients can thus avail themselves of cloud services provided by a cloud service provider without having to purchase separate licenses, support, or hardware and software resources for the services. For example, a cloud service provider's system may host an application, and a user may, via a network 1125 (e.g., the internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation® of Redwood Shores, California, such as middleware services, database services, Java cloud services, and others.

In certain aspects, cloud infrastructure system 1105 may provide one or more cloud services using different models, such as under a Software as a Service (SaaS) model, a Platform as a Service (PaaS) model, an Infrastructure as a Service (IaaS) model, and others, including hybrid service models. Cloud infrastructure system 1105 may include a suite of applications, middleware, databases, and other resources that enable provision of the various cloud services.

A SaaS model enables an application or software to be delivered to a client over a communication network like the Internet, as a service, without the client having to buy the hardware or software for the underlying application. For example, a SaaS model may be used to provide clients access to on-demand applications that are hosted by cloud infrastructure system 1105. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a client as a cloud service to provide elastic compute and storage capabilities. Various IaaS services are provided by Oracle Corporation®.

A PaaS model is generally used to provide, as a service, platform and environment resources that enable clients to develop, run, and manage applications and services without the client having to procure, build, or maintain such resources. Examples of PaaS services provided by Oracle Corporation® include, without limitation, Oracle Java Cloud Service (JCS), Oracle Database Cloud Service (DBCS), data management cloud service, various application development solutions services, and others.

Cloud services are generally provided on an on-demand self-service basis, subscription-based, elastically scalable, reliable, available, and secure manner. For example, a client, via a subscription order, may order one or more services provided by cloud infrastructure system 1105. Cloud infrastructure system 1105 then performs processing to provide the services requested in the client's subscription order. Cloud infrastructure system 1105 may be configured to provide one or even multiple cloud services.

Cloud infrastructure system 1105 may provide cloud services via different deployment models. In a public cloud model, cloud infrastructure system 1105 may be owned by a third-party cloud services provider and the cloud services are offered to any general public client, where the client can be an individual or an enterprise. In certain other aspects, under a private cloud model, cloud infrastructure system 1105 may be operated within an organization (e.g., within an enterprise organization) and services provided to clients that are within the organization. For example, the clients may be various departments of an enterprise, such as the Human Resources department, the payroll department, etc. or even individuals within the enterprise. In certain other aspects, under a community cloud model, the cloud infrastructure system 1105 and the services provided may be shared by several organizations in a related community. Various other models, such as hybrids of the above-mentioned models may also be used.

Client devices 1111, 1115, and 1120 may be of several types (such as client computing devices 1005, 1010, 1015, and 1020 depicted in FIG. 10) and may be capable of operating one or more client applications. A user may use a client device to interact with cloud infrastructure system 1105, such as to request a service provided by cloud infrastructure system 1105. For instance, a user might employ a client device to execute real-time data querying operations within the cloud. A client may use a client device, such as a laptop to interact with the subject ranking system integrated within cloud infrastructure system. The client may request GPU-accelerated computing instances of the cloud for training deep learning models. The cloud may provide the necessary resources, and the patient client may monitor and manage the training process through the laptop. Upon completion, the client may retrieve the trained models and results.

In certain aspects, to facilitate efficient provisioning of these resources for supporting the various cloud services provided by cloud infrastructure system 1105 for different clients, the resources may be bundled into sets of resources or resource modules. Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1105 may comprise multiple subsystems. These subsystems may be implemented in software, or hardware, or combinations thereof. As depicted in FIG. 11, the subsystems may include a user interface subsystem 1130 that enables users or clients of cloud infrastructure system 1105 to interact with cloud infrastructure system 1105. User interface subsystem 1130 may include various interfaces, such as a web user interface 1135, an online store interface 1140 where cloud services provided by cloud infrastructure system 1105 are advertised and are purchasable by a consumer, and other interfaces 1145. For example, a client may, using a client device, request (service request 1175) one or more services provided by cloud infrastructure system 1105 using one or more of interfaces 1135, 1140, and 1145. For example, a client may access the online store, browse cloud services offered by cloud infrastructure system 1105, and place a subscription order for one or more services offered by cloud infrastructure system 1105 that the client wishes to subscribe to. The service request may include information identifying the client and one or more services that the client desires to subscribe to. For example, a client may place a subscription order for a Chabot related service offered by cloud infrastructure system 1105. As part of the order, the client may provide information identifying for input (e.g., utterances).

In certain aspects, such as the illustrative example depicted in FIG. 11, cloud infrastructure system 1105 may comprise an order management subsystem (OMS) 1150 that is configured to process the new order. As part of this processing, OMS 1150 may be configured to: create an account for the client, if not done already; receive billing and/or accounting information from the client that is to be used for billing the client for providing the requested service to the client; verify the client information; upon verification, book the order for the client; and orchestrate various workflows to prepare the order for provisioning.

Once properly validated, OMS 1150 may then invoke the order provisioning subsystem (OPS) 1155 that is configured to provision resources for the order including processing, memory, and networking resources. The provisioning may include allocating resources for the order and configuring the resources to facilitate the service requested by the client order. The way resources are provisioned for an order and the type of the provisioned resources may depend upon the type of cloud service that has been ordered by the client. For example, according to one workflow, OPS 1155 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the requesting client for providing the requested service.

Cloud infrastructure system 1105 may itself internally use services 1170 that are shared by different components of cloud infrastructure system 1105 and which facilitate the provisioning of services by cloud infrastructure system 1105. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like. As depicted in the illustrative example in FIG. 11, cloud infrastructure system 1105 may include infrastructure resources 1165 that can be utilized for facilitating the provision of various cloud services offered by cloud infrastructure system 1105. Infrastructure resources 1165 may include, for example, processing resources, storage or memory resources, networking resources, and the like. Cloud infrastructure system 1105 may send a response or notification 1180 to the requesting client to indicate when the requested service is now ready for use. In some instances, information (e.g., a link) may be sent to the client that enables the client to start using and availing the benefits of the requested services.

Cloud infrastructure system 1105 may provide services to multiple clients in parallel. Cloud infrastructure system 1105 may store information for these clients, including possibly proprietary information. In certain aspects, cloud infrastructure system 1105 comprises an identity management subsystem (IMS) 1160 that is configured to manage client's information and provide the separation of the managed information such that information related to one client is not accessible by another client. IMS 1160 may be configured to provide various security-related services, such as identity services, such as information access management, authentication and authorization services, services for managing client identities and roles and related capabilities, and the like.

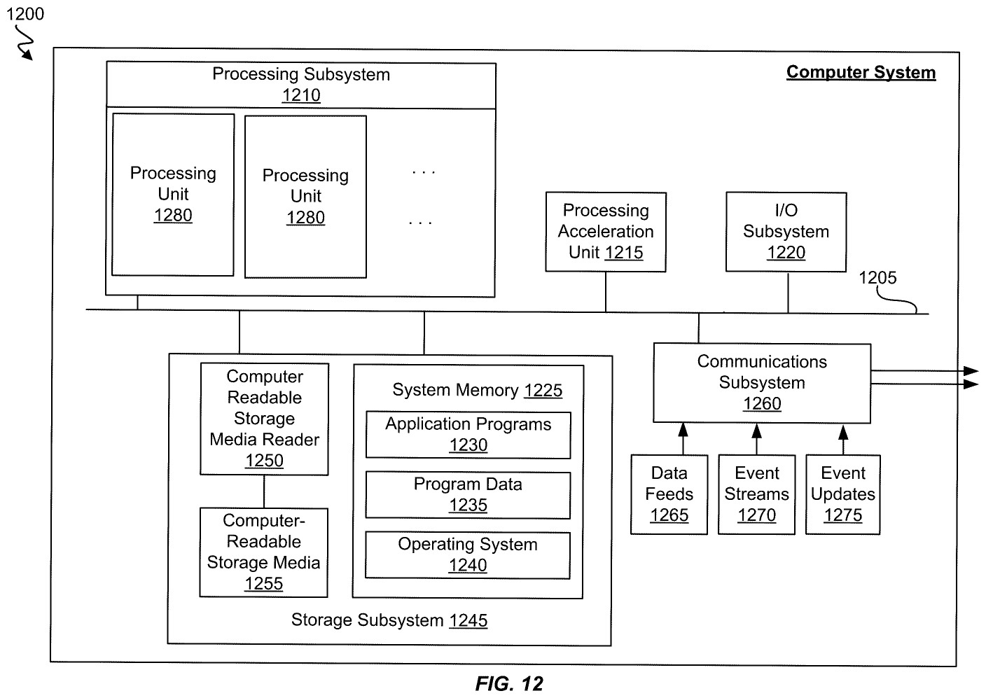

FIG. 12 illustrates an exemplary computer system 1200 that may be used to implement certain aspects of the present disclosure. For example, a computer system 1200 may facilitate the integration of a subject ranking system with the cloud by provisioning and configuring resources, managing data, implementing security measures, monitoring performance, and enabling scalability. It may serve as the foundational infrastructure, enabling seamless deployment and operation of AI applications within the cloud environment while providing flexibility and scalability to adapt to changing computational demands efficiently. In some aspects, computer system 1200 may be used to implement various servers as described above. As shown in FIG. 12, computer system 1200 may include various subsystems including a processing subsystem 1210 that communicates with a few other subsystems via a bus subsystem 1205. These other subsystems may include a processing acceleration unit 1215, an I/O subsystem 1220, a storage subsystem 1245, and a communications subsystem 1260. Storage subsystem 1245 may include non-transitory computer-readable storage media including storage media 1255 and a system memory 1225.

Bus subsystem 1205 provides a mechanism for letting the various components and subsystems of computer system 1200 communicate with each other as intended. Although bus subsystem 1205 is shown schematically as a single bus, alternative aspects of the bus subsystem may utilize multiple buses. Bus subsystem 1205 may be any of several types of bus structures including a memory bus or memory controller, a peripheral bus, a local bus using any of a variety of bus architectures, and the like. For example, such architectures may include an industry standard architecture (ISA) bus, micro channel architecture (MCA) bus, enhanced ISA (EISA) bus, video electronics standards association (VESA) local bus, and peripheral component interconnect (PCI) bus, which can be implemented as a Mezzanine bus manufactured to the IEEE P1286.1 standard, and the like.

Processing subsystem 1210 controls the operation of distributed system 1000 and may comprise one or more processors, application specific integrated circuits (ASICs), or field programmable gate arrays (FPGAs). The processors may include single core or multicore processors. The processing resources of computer system 1200 can be organized into one or more processing units 1280, 1280, etc. A processing unit may include one or more processors, one or more cores from the same or different processors, a combination of cores and processors, or other combinations of cores and processors. In some aspects, processing subsystem 1210 can include one or more special purpose co-processors, such as graphics processors, digital signal processors (DSPs), or the like. In some aspects, some or all of the processing units of processing subsystem 1210 can be implemented using customized circuits, such as application specific integrated circuits (ASICs), or field programmable gate arrays (FPGAs).

In some aspects, the processing units in processing subsystem 1210 can execute instructions stored in system memory 1225 or on computer readable storage media 1255. In various aspects, the processing units can execute a variety of programs or code instructions and can maintain multiple concurrently executing programs or processes. At any given time, some, or all of the program code to be executed can be resident in system memory 1225 and/or on computer-readable storage media 1255 including potentially on one or more storage devices. Through suitable programming, processing subsystem 1210 can provide various functionalities described above. In instances where computer system 1200 is executing one or more virtual machines, one or more processing units may be allocated to each virtual machine.

In certain aspects, a processing acceleration unit 1215 may optionally be provided for performing customized processing or for off-loading some of the processing performed by processing subsystem 1210 to accelerate the overall processing performed by computer system 1200.

I/O subsystem 1220 may include devices and mechanisms for inputting information to computer system 1200 and/or for outputting information from or via computer system 1200. In general, use of the term input device is intended to include all possible types of devices and mechanisms for inputting information to computer system 1200. User interface input devices may include, for example, a keyboard, pointing devices, such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices, such as the Microsoft Kinect® motion sensor that enables users to control and interact with an input device, the Microsoft Xbox® 360 game controller, devices that provide an interface for receiving input using gestures and spoken commands. User interface input devices may also include eye gesture recognition devices, such as the Google Glass® blink detector that detects eye activity (e.g., “blinking” while taking pictures and/or making a menu selection) from users and transforms the eye gestures as inputs to an input device (e.g., Google Glass®). Additionally, user interface input devices may include voice recognition sensing devices that enable users to interact with voice recognition systems (e.g., Siri® navigator) through voice commands.

Other examples of user interface input devices include, without limitation, three dimensional (3D) mice, joysticks or pointing sticks, gamepads and graphic tablets, and audio/visual devices, such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices, such as computed tomography, magnetic resonance imaging, position emission tomography, and medical ultrasonography devices. User interface input devices may also include, for example, audio input devices, such as MIDI keyboards, digital musical instruments, and the like.

In general, use of the term output device is intended to include all possible types of devices and mechanisms for outputting information from computer system 1200 to a user or other computer. User interface output devices may include a display subsystem, indicator lights, or non-visual displays, such as audio output devices, etc. The display subsystem may be a cathode ray tube (CRT), a flat-panel device, such as that using a liquid crystal display (LCD) or plasma display, a projection device, a touch screen, and the like. For example, user interface output devices may include, without limitation, a variety of display devices that visually convey text, graphics, and audio/video information, such as monitors, printers, speakers, headphones, automotive navigation systems, plotters, voice output devices, and modems.

Storage subsystem 1245 provides a repository or data store for storing information and data that is used by computer system 1200. Storage subsystem 1245 provides a tangible non-transitory computer-readable storage medium for storing the basic programming and data constructs that provide the functionality of some aspects. Storage subsystem 1245 may store software (e.g., programs, code modules, instructions) that when executed by processing subsystem 1210 provides the functionality described above. The software may be executed by one or more processing units of processing subsystem 1210. Storage subsystem 1245 may also provide a repository for storing data used in accordance with the teachings of this disclosure.

Storage subsystem 1245 may include one or more non-transitory memory devices, including volatile and non-volatile memory devices. As shown in FIG. 12, storage subsystem 1245 includes a system memory 1225 and a computer-readable storage media 1255. System memory 1225 may include a number of memories including a volatile main random-access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 1200, such as during start-up, may typically be stored in the ROM. The RAM typically contains data and/or program modules that are presently being operated and executed by processing subsystem 1210. In some implementations, system memory 1225 may include multiple different types of memory, such as static random-access memory (SRAM), dynamic random-access memory (DRAM), and the like.

By way of example, and not limitation, as depicted in FIG. 12, system memory 1225 may load application programs 1230 that are being executed, which may include various applications, such as Web browsers, mid-tier applications, relational database management systems (RDBMS), etc., program data 1235, and an operating system 1240. By way of example, operating system 1240 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems, such as iOS, Windows® Phone, Android® OS, BlackBerry® OS, Palm® OS operating systems, and others.

Computer-readable storage media 1255 may store programming and data constructs that provide the functionality of some aspects. Computer-readable media 1255 may provide storage of computer-readable instructions, data structures, program modules, and other data for computer system 1200. Software (programs, code modules, instructions) that, when executed by processing subsystem 1210 provides the functionality described above, may be stored in storage subsystem 1245. By way of example, computer-readable storage media 1255 may include non-volatile memory, such as a hard disk drive, a magnetic disk drive, an optical disk drive, such as a CD ROM, digital video disc (DVD), a Blu-Ray® disk, or other optical media. Computer-readable storage media 1255 may include, but is not limited to, Zip® drives, flash memory cards, universal serial bus (USB) flash drives, secure digital (SD) cards, DVD disks, digital video tape, and the like. Computer-readable storage media 1255 may also include, solid-state drives (SSD) based on non-volatile memory, such as flash-memory based SSDs, enterprise flash drives, solid state ROM, and the like, SSDs based on volatile memory, such as solid state RAM, dynamic RAM, static RAM, dynamic random access memory (DRAM)-based SSDs, magneto resistive RAM (MRAM) SSDs, and hybrid SSDs that use a combination of DRAM and flash memory based SSDs.

In certain aspects, storage subsystem 1245 may also include a computer-readable storage media reader 1250 that can further be connected to computer-readable storage media 1255. The computer-readable storage media reader 1250 may receive and be configured to read data from a memory device, such as a disk, a flash drive, etc.

In certain aspects, computer system 1200 may support virtualization technologies, including but not limited to virtualization of processing and memory resources. For example, computer system 1200 may provide support for executing one or more virtual machines. In certain aspects, computer system 1200 may execute a program, such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1200. Accordingly, multiple operating systems may potentially be run concurrently by computer system 1200.

Communications subsystem 1260 provides an interface to other computer systems and networks. Communications subsystem 1260 serves as an interface for receiving data from and transmitting data to other systems from computer system 1200. For example, communications subsystem 1260 may enable computer system 1200 to establish a communication channel to one or more client devices via the Internet for receiving and sending information from and to the client devices. For example, the communication subsystem may be used to transmit a response to a user regarding the inquiry for a Chatbot.

Communication subsystem 1260 may support both wired and/or wireless communication protocols. For example, in certain aspects, communications subsystem 1260 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects, communications subsystem 1260 can provide wired network connectivity (e.g., Ethernet) in addition to or instead of a wireless interface.

Communication subsystem 1260 can receive and transmit data in various forms. For example, in some aspects, in addition to other forms, communications subsystem 1260 may receive input communications in the form of data feeds 1265 such as structured and/or unstructured data feeds, event streams 1270, event updates 1275, and the like. For example, communications subsystem 1260 may be configured to receive (or send) data feeds 1265 in real-time from users of social media networks and/or other communication services, such as Twitter® feeds, Facebook® updates, web feeds, such as Rich Site Summary (RSS) feeds, and/or real-time updates from one or more third party information sources.

In certain aspects, communications subsystem 1260 may be configured to receive data in the form of continuous data streams, which may include event streams 1270 of real-time events and/or event updates 1275, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1260 may also be configured to communicate data from computer system 1200 to other computer systems or networks. The data may be communicated in various forms, such as structured and/or unstructured data feeds 1265, event streams 1270, event updates 1275, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a personal digital assistant (PDA)), a wearable device (e.g., a Google Glass® head mounted display), a personal computer, a workstation, a mainframe, a kiosk, a server rack, or any other data processing system. Due to the ever-changing nature of computers and networks, the description of computer system 1200 depicted in FIG. 12 is intended only as a specific example. Many other configurations having more or fewer components than the system depicted in FIG. 12 are possible. Based on the disclosure and teachings provided herein, a person of ordinary skill in art can appreciate other ways and/or methods to implement the various aspects.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
identifying a set of subjects flagged for engagement in a potential communication workflow;
for each subject of the set of subjects:
accessing subject data that identifies one or more historical medical events or one or more characteristics of a subject;
generating one or more probabilities, by leveraging one or more causal models, wherein the one or more probabilities include:
a first probability associated with a positive outcome if an intervention is performed,
a second probability associated with a positive outcome in an absence of the intervention, and
a third probability representing a likelihood of the intervention being performed under standard care;
generating, using a first mathematical function, a net-benefit bound by combining the one or more probabilities;
predicting, via one or more machine-learning models, one or more costs and revenue amounts including:
a cost associated with performing the intervention,
a cost associated with an acute-care utilization or an emergency department visit due to a negative outcome, and
a revenue generated from closing care gaps; and
computing, using a second mathematical function, a net-cost saving bound based on the net-benefit bound and the one or more costs and revenue amounts;
ranking the set of subjects based on the net-cost saving bounds to generate a ranked list; and
initiating one or more preventative measures for one or more subjects of the set of subjects having a high ranking in the ranked list, wherein the one or more preventative measures include scheduling follow-up appointments or checkups, calling the subject for additional diagnostic lab tests, or providing personalized healthcare recommendations.

2. The computer-implemented method of claim 1, wherein the one or more causal models include directed acyclic graphs (DAGs), inverse probability weighing (IPW), uplift modeling, propensity score matching (PSM), structural causal modeling (SCM), or structural equation modeling (SEM).

3. The computer-implemented method of claim 2, wherein the one or more causal models are based on one or more binary classifiers implemented via a gradient-boosting technique for predicting the first probability, the second probability, and the third probability.

4. The computer-implemented method of claim 1, wherein the one or more costs and revenue amounts are predicted using one or more predictive models including a regression model, a neural network, a Bayesian model, or a reinforcement learning-based approach.

5. The computer-implemented method of claim 1, wherein ranking the set of subjects further comprises applying one or more ranking techniques to generate the ranked list based on the net-cost saving bounds, wherein the one or more ranking techniques include: modified competition ranking, dense ranking, ordinal ranking, standard competition ranking, fractional ranking, Bayesian ranking, RankNet, or XBoostRanker.

6. The computer-implemented method of claim 1, wherein the net-benefit bound includes an upper and a lower bound quantifying a likelihood of the subject experiencing a positive outcome from the intervention, and wherein the upper and the lower bounds represent a range of potential benefit estimates.

7. The computer-implemented method of claim 1, wherein the positive outcome corresponds to an occurrence of a target outcome resulting from the intervention, and wherein the negative outcome corresponds to an occurrence of an outcome opposite to the target outcome from the intervention.

8. The computer-implemented method of claim 1, wherein the subject data includes attributes including age, vitals, lab results, prescriptions, previous admissions and/or clinical history.

9. A system comprising:
one or more data processors; and
a non-transitory computer-readable storage medium containing instructions which, when executed on the one or more data processors, cause the one or more data processors to perform a set of operations including:
identifying a set of subjects flagged for engagement in a potential communication workflow;
for each subject of the set of subjects:
accessing subject data that identifies one or more historical medical events or one or more characteristics of a subject;
generating one or more probabilities, by leveraging one or more causal models, wherein the one or more probabilities include:
a first probability associated with a positive outcome if an intervention is performed,
a second probability associated with a positive outcome in an absence of the intervention, and
a third probability representing a likelihood of the intervention being performed under standard care;
generating, using a first mathematical function, a net-benefit bound by combining the one or more probabilities;
predicting, via one or more machine-learning models, one or more costs and revenue amounts including:
a cost associated with performing the intervention,
a cost associated with an acute-care utilization or an emergency department visit due to a negative outcome, and
a revenue generated from closing care gaps; and
computing, using a second mathematical function, a net-cost saving bounds based on the net-benefit bound and the one or more costs and revenue amounts;
ranking the set of subjects based on the net-cost saving bound to generate a ranked list; and
initiating one or more preventative measures for one or more subjects of the set of subjects having a high ranking in the ranked list, wherein the one or more preventative measures include scheduling follow-up appointments or checkups, calling the subject for additional diagnostic lab tests, or providing personalized healthcare recommendations.

10. The system of claim 9, wherein the one or more causal models include directed acyclic graphs (DAGs), inverse probability weighing (IPW), uplift modeling, propensity score matching (PSM), structural causal modeling (SCM), or structural equation modeling (SEM).

11. The system of claim 10, wherein the one or more causal models are based on one or more binary classifiers implemented via a gradient-boosting technique for predicting the first probability, the second probability, and the third probability.

12. The system of claim 9, wherein the one or more costs and revenue amounts are predicted using one or more predictive models including a regression model, a neural network, a Bayesian model, or a reinforcement learning-based approach.

13. The system of claim 9, wherein ranking the set of subjects further comprises applying one or more ranking techniques to generate the ranked list based on the net-cost saving bounds, wherein the one or more ranking techniques include: modified competition ranking, dense ranking, ordinal ranking, standard competition ranking, fractional ranking, Bayesian ranking, RankNet, or XBoostRanker.

14. The system of claim 9, wherein the net-benefit bound includes an upper and a lower bound quantifying a likelihood of the subject experiencing a positive outcome from the intervention, and wherein the upper and the lower bounds represent a range of potential benefit estimates.

15. The system of claim 9, wherein the positive outcome corresponds to an occurrence of a target outcome resulting from the intervention, and wherein the negative outcome corresponds to an occurrence of an outcome opposite to the target outcome from the intervention.

16. A computer-program product tangibly embodied in a non-transitory machine-readable storage medium, including instructions configured to cause one or more data processors to perform a set of operations comprising:
identifying a set of subjects flagged for engagement in a potential communication workflow;
for each subject of the set of subjects:
accessing subject data that identifies one or more historical medical events or one or more characteristics of a subject;
generating one or more probabilities, by leveraging one or more causal models, wherein the one or more probabilities include:
a first probability associated with a positive outcome if an intervention is performed,
a second probability associated with a positive outcome in an absence of the intervention, and
a third probability representing a likelihood of the intervention being performed under standard care;
generating, using a first mathematical function, a net-benefit bound by combining the one or more probabilities;
predicting, via one or more machine-learning models, one or more costs and revenue amounts including:
a cost associated with performing the intervention,
a cost associated with an acute-care utilization or an emergency department visit due to a negative outcome, and
a revenue generated from closing care gaps; and
computing, using a second mathematical function, a net-cost saving bound based on the net-benefit bound and the one or more costs and revenue amounts;
ranking the set of subjects based on the net-cost saving bound to generate a ranked list; and
initiating one or more preventative measures for one or more subjects of the set of subjects having a high ranking in the ranked list, wherein the one or more preventative measures include scheduling follow-up appointments or checkups, calling the subject for additional diagnostic lab tests, or providing personalized healthcare recommendations.

17. The computer-program product of claim 16, wherein the one or more causal models include directed acyclic graphs (DAGs), inverse probability weighing (IPW), uplift modeling, propensity score matching (PSM), structural causal modeling (SCM), or structural equation modeling (SEM).

18. The computer-program product of claim 17, wherein the one or more costs and revenue amounts are predicted using one or more predictive models including a regression model, a neural network, a Bayesian model, or a reinforcement learning-based approach.

19. The computer-program product of claim 16, wherein ranking the set of subjects further comprises applying one or more ranking techniques to generate the ranked list based on the net-cost saving bounds, wherein the one or more ranking techniques include: modified competition ranking, dense ranking, ordinal ranking, standard competition ranking, fractional ranking, Bayesian ranking, RankNet, or XBoostRanker.

20. The computer-program product of claim 16, wherein the net-benefit bound includes an upper and a lower bound quantifying a likelihood of the subject experiencing a positive outcome from the intervention, and wherein the upper and lower the bounds represent a range of potential benefit estimates.
